(19) United States
(12) Patent Application Publication
(10) Pub. No.: US 2026/0161012 A1
(43) Pub. Date: Jun. 11, 2026

(54) VIRTUAL DISPLAY SYSTEMS IN MEDICAL DIAGNOSTIC TOOLS

(71) Applicant: BRELYON, Inc., San Mateo, CA (US)

(72) Inventors: Barmak Heshmat Dehkordi, San Mateo, CA (US); Christopher Barsi, Lee, NH (US)

(73) Assignee: BRELYON, Inc., San Mateo, CA (US)

(21) Appl. No.: 19/300,906

(22) Filed: Aug. 15, 2025

Related U.S. Application Data
(60) Provisional application No. 63/689,096, filed on Aug. 30, 2024.

(51) Int. Cl. G02B 30/40; A61B 5/00

(52) U.S. Cl. CPC G02B 30/40; A61B 5/742

(57) ABSTRACT

Systems and methods of virtual displays integrated into medical and other environments, in particular in medical diagnostic tools or medical imaging tools. In some embodiments, the viewer is close to an exit aperture of the display system. In some embodiments, components and subsystems of the apparatus are non-magnetic and compatible with MRI in-bore applications. In some embodiments, the light source is physically distant or exterior from the optical subsystem producing a virtual image. In some embodiments, the virtual image has monocular depth or stereoscopic depth cues.

This application claims priority under 35 U.S.C. § 119(e) to U.S. provisional patent application, U.S. Ser. No. 63/689,096, filed on Aug. 30, 2024, which is herein incorporated by reference in its entirety.

### FIELD OF THE INVENTION

The present invention relates to display systems for integration into existing systems. The systems include medical diagnostic tools or medical imaging devices, such as in-bore MRI architectures to provide visually ergonomic imagery to mitigate claustrophobia.

### BACKGROUND

Increasing movement towards more immersive lightfield and/or autostereoscopic three-dimensional (3D) displays is due to advancement in electronics and microfabrication. 3D display technologies, such as virtual reality (VR) and augmented reality (AR) headsets, are often interested in presenting to a viewer an image that is perceived at a depth far behind the display device itself. Refractive elements can produce such an image but suffer from increased bulk and optical aberrations. Further, such displays may cause eye strain, nausea, or other fatigue symptoms.

Virtual display systems are designed and implemented with various specifications. For example, in U.S. Pat. Nos. 11,067,825 B2 and 11,768,825 B1, Dehkordi described a virtual display system providing monocular and binocular depth cues to achieve realistic depth perception effects. In U.S. Pat. No. 11,592,684 B2, Dehkordi disclosed an optical component called a field evolving cavity to make the light source appear farther from the viewer compared to the distance to the physical display system. In U.S. Pat. No. 11,196,976 B2, Dehkordi further disclosed a virtual display system directed to tessellating a light field to extend beyond the pupil size of a display system. In U.S. Pat. No. 11,662,591 B1, Dehkordi et al disclosed an apparatus for modifying the monocular depth of virtual images dynamically and for producing a multifocal virtual image. Last, in U.S. Pat. No. 11,320,668 B2, Dehkordi et al disclosed a method of modifying the optical quality or the properties of a display system using optical fusion, which combines computational methods with optical architectures to remove visual artifacts from the images produced by the display system.

### SUMMARY

Some aspects of the present invention relate to display systems for use in medical diagnostic tools. In certain embodiments, a virtual display system is mechanically secured to an interior or an exterior surface of a medical diagnostic tool such as an MRI machine, such that the headbox where the virtual image is visible is seen by a patient within the bore of the MRI. In some embodiments, the mechanical coupling between the display system and the medical diagnostic tool is through one or more mechanical supports, which may be coupled to each other, the medical diagnostic tool, or the display system through articulated joints. The patient viewing the virtual image is triggered to focus his eyes to accommodate a monocular depth that lies outside the bore, i.e., the virtual image lays farther from the patient than the distance between the patient and the bore or cavity. The virtual image has a lateral size that also generates a spacious viewing environment. The image content and specifications therefore alleviate claustrophobic effects during the imaging process.

The materials out of which the optical components—including entrance aperture optics, exit aperture optics, and light-guiding subsystems or depth modulation subsystems (such as a field evolving cavity)—and any mechanical housings, joints, or supports are made are such that the resulting display system does not interfere with the imaging process, does not pose harm to the patient, does not damage the medical diagnostic tool, and does not cause spurious diagnostics. In the case of an MRI machine, the materials are non-magnetic, appropriately shielded, and/or non-ferrous materials.

In some embodiments, a system comprises a light guiding subsystem including a plurality of specular reflectors, a housing to secure the light guiding subsystem, an entrance aperture in the housing to direct light from an external light source into the light guiding subsystem, and an exit aperture in the housing to direct light into a headbox, wherein the specular reflectors are non-magnetic, and the light forms a virtual image visible within the headbox.

In some embodiments of the system, the light source is greater than 0.5 meters from the entrance aperture.

In some embodiments, the system further comprises a mechanical joint to mount the housing to a medical imaging device.

In some embodiments of the system, the medical imaging device is a magnetic resonance imaging (MRI) system, the housing positioned inside a bore of the MRI system.

In some embodiments of the system, the mechanical joint is a track.

In some embodiments, the system further comprises the light source.

In some embodiments of the system, the headbox spans a lateral dimension of at least 10 cm.

In some embodiments of the system, at least one specular reflector among the plurality of specular reflectors comprises a multilayer dielectric film.

In some embodiments of the system, at least one specular reflector among the plurality of specular reflectors is a curved specular reflector.

In some embodiments of the system, a subset of the plurality of specular reflectors is polarization dependent.

In some embodiments, the system further comprises an aperture optic disposed at the exit aperture.

In some embodiments, the system further comprises an entrance aperture optic disposed at the entrance aperture.

In some embodiments of the system, a monocular depth is at least 1.5 times a distance between the headbox and the exit aperture.

In some embodiments, the system further comprises a collimating optic to collect the light from the light source onto the entrance aperture.

In some embodiments, the system further comprises a liquid crystal plate disposed along a path of the light.

In some embodiments of the system, the light source is selected from a group including a laser, an LED, a laser projection system, a display panel, and combinations thereof.

In some embodiments of the system, the mechanical joint is adjustable, such that a position of the headbox is variable.

In some embodiments of the system, the image is a multifocal image.

In some embodiments of the system, the image comprises a left image and a right image to generate a stereoscopic depth cue.

In some embodiments of the system, the curved specular reflector is located at the back of the display, and the light is incident on a second specular reflector at least twice.

In some embodiments of the system, at least one specular reflector among the specular reflector comprises a glass substrate with a non-magnetic coating.

In some embodiments of the system, at least one specular reflector among the plurality of specular reflectors is plastic.

In some embodiments of the system, a lateral size of the virtual image is greater than a lateral physical dimension of the exit aperture.

In some embodiments of the system, the lateral size of the virtual image is at least two times greater than the lateral physical dimension of the exit aperture.

In some embodiments of the system, at least one specular reflector among the plurality of specular reflectors comprises a material selected from a group consisting of carbon fiber, silicone, gelatin, polymer, and combinations thereof.

In some embodiments of the system, at least one specular reflector among the plurality of specular reflector comprises a nonferrous metal.

In some embodiments, the system further comprises a reflector external to the housing to receive the light and redirect it to the entrance aperture.

In some embodiments of the system, the housing is secured to an external surface of a medical imaging system.

In some embodiments of the system, wherein at least one specular reflector among the plurality of specular reflectors is curved.

In some embodiments, a system comprises a light guiding subsystem including a diffractive waveguide and a grating, a housing to secure the light guiding subsystem, an entrance aperture in the housing to direct light from an external light source into the light guiding subsystem, and an exit aperture in the housing to direct light into a headbox, wherein the specular reflectors are non-magnetic, and the light forms a virtual image visible within the headbox.

In some embodiments, the system further comprises reflector external to the housing to receive the light and redirect it to the entrance aperture.

In some embodiments of the system, the grating is a surface grating disposed at the exit aperture.

In some embodiments of the system, the waveguide is a plurality of waveguides, each waveguide among the plurality of waveguides accepting light of a respective spectral range.

In some embodiments of the system, the grating is a plurality of waveguides, coupled to a respective waveguide.

In some embodiments, the system further comprises a non-magnetic holographic mirror.

In some embodiments, a system comprises a field evolving (FEC) cavity having a non-magnetic reflector and a non-magnetic semi-reflector, a housing to secure the FEC, an entrance aperture in the housing to direct light from an external light source into the light guiding subsystem, and an exit aperture in the housing to direct light into a headbox, wherein the light forms a virtual image visible within the headbox.

In some embodiments of the system, the housing is secured to a medical diagnostic tool.

In some embodiments of the system, the housing is secured to a mechanical joint to couple the housing to a medical diagnostic tool.

In some embodiments, the system further comprises an entrance optic disposed at the entrance aperture, the entrance optic.

In some embodiments, the system further comprises an exit aperture optic disposed at the exit aperture.

### BRIEF DESCRIPTION OF THE DRAWINGS

FIG. 1 illustrates common features, components, or elements of the disclosed embodiments.

FIGS. 2A through 2C depict a set of subsystems and optical architectures using the components of FIG. 1.

FIGS. 3A through 3D show perspective views of the disclosed invention as integrated into a diagnostic imaging too.

FIG. 4 depicts a flow diagram to explain the functionality of the different structures and subsystems disclosed herein.

FIGS. 5A through 5I show a set of embodiments of display systems, including the light-guiding subsystems to generate virtual images with monocular depth.

FIGS. 6A through 6L show a set of embodiments to illustrate how the display system is integrable into a medical diagnostic tool and how the display system's geometry may be articulated.

FIGS. 7A through 7F show a set of possible image-forming light sources to use with the display system.

FIGS. 8A through 8D show auxiliary embodiments of the display system that include remote sensing, stereoscopic imagery, and multifocal imagery.

### DETAILED DESCRIPTION

For example, mechanisms incorporate such content into a variety of display systems that include, but are not limited to, three-dimensional displays, virtual and multilayer displays, or even multi-monitor setups. In some embodiments, the display images are just 2D images extended to side panels and monitors. In some other embodiments, the display provides images with monocular depth, wherein a viewer experiences accommodation depth cues to at least one image plane. In some embodiments, the display images are stereoscopic images. In some embodiments, both stereoscopic and monocular depth cues are provided.

The embodiments of the display system disclosed herein produce virtual images that are both comfortable to view and mitigate the effects of claustrophobia in small viewing spatial environments, such as in the bore of an MRI machine. However, such an example is not meant to be limiting, and the size of the display system and its components can be engineered for similar applications in different medical, scientific, or generically space-restrictive environments.

### Nomenclature

In this description, references to an “embodiment,” “one embodiment,” or similar words or phrases mean that the feature, function, structure, or characteristic being described is an example of the technique or invention introduced here. Occurrences of such phrases in this specification do not necessarily all refer to the same embodiment. On the other hand, the embodiments referred to herein also are not necessarily mutually exclusive. The invention here is explained relative to preferred embodiments, but it is to be understood that modifications or variations can be made without departing from the scope of the claimed invention.

All references to “user,” “users,” “observer,” or “viewer,” pertain to either an individual or individuals who would use the apparatus, methods, and techniques introduced here. A user interacts with a system using a sense, which could be visual, auditory, tactile, or olfactory. In some embodiments, the system is a display system, and the user or viewer is viewing the image content. A user may be a future or past user to allow for asynchronous applications.

The term “arbitrarily engineered” means being of any shape, size, material, feature, type or kind, orientation, location, quantity, components, and arrangement of single components or arrays of components that enables the present invention. Two elements are “optically coupled” when the first element being imparts, transfers, feeds, or directs light to the second element directly or indirectly. More generally, two elements are “coupled” when the first element being imparts, transfers, feeds, or directs energy or information to the second element directly or indirectly. The energy may be light, acoustic, thermal, electronic, mechanical, radio-frequency or other electromagnetic energy, and the like. The information includes any structure of the energy forming data.

In this disclosure, the “lightfield” at a plane refers to a vector field that describes the amount of light flowing in every or several selected directions through every point in that plane. The lightfield is the description of the angles and intensities of light rays traveling through or emitted from that plane. A “fractional lightfield” is a subsampled version of the lightfield such that full lightfield vector field is represented by a finite number of samples in different focal planes and/or angles. Some lightfield models incorporate wave-based effects like diffraction. A lightfield display is a three-dimensional display that is designed to produce 3D effects for a user using lightfield modeling. The terms “concentric light field” or “curving light field” as used herein mean a lightfield for which for any first pixel and second pixel of the display at a fixed radius from the viewer, the chief ray of the light cone emitted from the first pixel in a direction perpendicular to the surface of the display intersects with the chief ray of the light cone emitted from the second pixel in a direction perpendicular to the surface of the display. A concentric lightfield produces an image that is focusable to the eye at all points, including pixels that are far from the optical axis of the system (the center of curvature), where the image is curved rather than flat, and the image is viewable within a specific viewing space (headbox) in front of the lightfield. As used herein, the term “chief ray” refers to the central axis of a light cone that is emitted by a pixel source or a point-like source, or that is reflected by a point on an object.

“Monocular optical depth” or “monocular depth” is the perceived distance, or apparent depth, between the observer and the apparent position of an image. It equals the distance to which an eye accommodates (focuses) to see a clear image. Thus, the monocular depth is the accommodation depth corresponding to the accommodation depth cue. Each eye experiences this depth cue.

A “virtual image” is an image that triggers a depth cue of a viewer, who consequently perceives display content at variable depths, different parts of the display content at various depths relative to each other, or display content that appears at a different depth than a distance between the viewer and a component of the physical display system. For example, some depth cues are parallax effects. In some embodiments, 3D effects are triggered stereoscopically by sending a different image to each eye corresponding to a disparity. In some embodiments, depth cues are triggered using monocular depth cues, wherein each eye focuses or accommodates to the appropriate monocular depth. Virtual images may be multifocal, varifocal, lightfield images, holographic, stereoscopic, autostereoscopic, or (auto)multi-scopic. The virtual depth of a virtual image may be dynamically adjustable via a control in the display system, a user or sensor input, or a pre-programmed routine.

Monocular depths may be understood as follows. A point source of light emits light rays equally in all directions, and the tips of these light rays can be visualized as all lying on a spherical surface, called a wavefront, of expanding radius. In geometric optics in, for example, free space or isotropic media, the wavefront is identical the surface that is everywhere perpendicular to the light rays, and can be calculated by e.g., the eikonal equation, Lagrangian optics, Hamiltonian optics, and the like. When the point source is moved farther from an observer, emitted light rays travel a longer distance to reach the observer and therefore their tips lie on a spherical wavefront of larger radius and correspondingly smaller curvature, i.e., the wavefront is flatter. This flatter wavefront is focused by an eye differently than a less flat one. Thus, the point source is perceived by an eye or camera as a farther distance, or deeper depth, to the object. Monocular depth does not require both eyes, or stereopsis, to be perceived. An extended object can be considered as a collection of point sources at varying positions and as consequently emitting a wavefront corresponding to the sum of the point-source wavefronts, so the same principles apply to, e.g., an illuminated object or emissive display panel. Wavefront evolution refers to changes in wavefront curvature due to optical propagation.

A virtual image is produced by a virtual display system, which produces images at two or more perceived depths, or a perceived depth that is different from the depth of the display panel that generates the image. A virtual display system may be a free-standing system, like a computer monitor or television set. It may also be part of a cellphone, tablet, headset, smart watch, or any portable device. It may be for a single user or multiple users in any application. Virtual display systems may be volumetric or lightfield displays, multifocal displays, and the like. In some embodiments, the virtual display system is a holographic display, which relies on the wave nature of light to produce images based on manipulating interference the light.

“Depth modulation” refers to the change, programming, or variation of the monocular depth of a virtual image.

A virtual image is to be viewed by an observer, rather than be projected directly onto a screen. The light forming the image has traveled an optical distance corresponding to the monocular depth at which a viewer perceives the image. The geometric plane in space in which the virtual image is located is called the “focal plane.” Concentric lightfield displays may produce curved focal planes. A virtual image comprising a set of virtual images at different focal planes is called a “multifocal image” or “multilayer image.” E.g., a multilayer display system is one in which display content is shown in such a way that a viewer must accommodate his eyes to different depths to see different display content. A virtual image whose focal plane can be adjusted dynamically, e.g., by varying an optical or electrical property of the display system, is also called a multifocal image. A virtual display system that produces multifocal images may be called a “multifocal display system,” “multilayer display system,” and the like. A monocular depth at which content is located is also called a “virtual depth,” or “focal plane.” Multilayer displays comprise transparent displays in some embodiments. Content at a given virtual depth may be called a “layer,” “depth layer,” “virtual layer,” and the like.

A display system may produce a real image in the space outside the display system. (A real image forms where the light rays physically intersect, such that a film placed at that location will record a (collection of) bright spot(s), corresponding to an image.) The light rays diverge beyond that intersection point, such that a viewer sees a virtual image. That virtual image is first formed as a real image and will appear to the viewer as floating, or hovering, in front of the display panel, at the location of the real image location. This image is called a “hovering real image.”

The term “display content” is used to describe the source information or the final image information that is perceived by a viewer.

In “eyebox” is the volume of space wherein a human eye may be located to view an image. In some embodiments, the virtual display system produces an eyebox whose volume is big enough to encompass both eyes of a viewer simultaneously. In another embodiment, the virtual display system produces a left eyebox and a right eyebox, configured for simultaneous viewing by the left and the right eye, respectively. The size and number of eyeboxes depends on the specific nature and design of the display. “Headbox” is the volume of space where a viewer's eyes may be positioned for an image to be visible. In some embodiments, the headbox is larger than the average interpupillary distance for a person, such that both eyes can be located within the headbox simultaneously. The virtual images disclosed herein are simultaneously visible by both eyes of a view. In some embodiments the headbox is large enough for a plurality of viewers to see a virtual image. In some embodiments, headbox and eyebox are used interchangeably.

When the headbox is big enough to encompass both eyes of a viewer, each point of the virtual image is visible by both eyes of the viewer, i.e., light rays from any given point of the virtual image enter both eyes simultaneously. To receive the virtual image, the viewer's eyes may be located anywhere within the headbox, which spans a lateral dimension. The lateral dimension may be, for example, at least 8 cm, at least 10 cm, at least 15 cm, at least 20 cm, or at least 30 cm. The distance between the display system and the nearest viewing position in the headbox may be, for example, between at least 30 and 60 cm, greater than 20 cm, or less than 100 cm. This distance is in part limited by the viewing direction required to see the virtual image.

Display systems may incorporate any hardware, including liquid crystals or other polarization-dependent elements to impact properties of the display; any type of mirror or lens to redirect the light path, influence the size in any dimension, modify the focal depth, or correct for aberrations and distortions; any surface coatings, active elements; spectral or spatial filters to assist in image quality; optical cavities; or any type of element or coating to serve as a shield layer or antireflection layer to reduce unwanted, stray, or ambient light from reaching a viewer. In some embodiments, display systems comprise metamaterials and metasurfaces, nonlinear optical elements, photonic crystals, graded-index materials, anisotropic or bi-anisotropic elements, or electro-optic elements. In some embodiments, display systems are virtual display systems. Further, display systems can be of any modality, including infrared (IR), mid-IR, near-IR, far-IR, ultraviolet (UV), terahertz (THz), radiofrequency, or acoustic or ultrasonic (for consumption by a person's human auditory or tactile senses). The displays, or elements of the display may be curved in some embodiments.

A display system can produce images, overlay annotations on existing images, feed one set of display content back into another set for an interactive environment, or adjust to environmental surroundings. Users may have VR, AR, MR, or XR experiences; video-see through effects; monitor remote systems and receive simultaneous predictive suggestions; provide an avatar with permissions to make imprints on digital content or online resources; or use AI for generative content creation. A subsection of the display content may be input into an algorithm to impact another subsection.

A “subsection” of display content is a partitioning of the display content produced by the display system. In some embodiments, a subsection is a pixel or set of pixels. The set of pixels may be disjoint or contiguous. In some embodiments, a subsection corresponds to a feature type of the display content. For example, a subsection of an image of a person may be a head or an arm, and another subsection may be a hand or an eye. In some embodiments, a subsection may be an entire layer or part of a layer or focal plane of a display that produces multiple focal planes. In some embodiments, a subsection is a part of the spectral content of an image or a portion of the image in an arbitrary mathematical basis. Subsections may also be partitioned differently at various times. In some embodiments, a subsection is one of the segments of a segmented display.

Display content may be manipulated by a user or interactive with a user through various input devices. Input devices are types of sensors that take in a user input, usually deliberately rather than automatically. Input devices, such as cameras, keyboard and mouse input, touch screens, gesture sensors, head tracking, eye tracking, VR paddles, sound input, speech detection, allow for user feedback in multiple modalities. In some embodiments, various biological or health sensors capture information—such as heart rate, posture, seating or standing orientation, blood pressure, eye gaze or focus—and use that information in an algorithm to influence or impact the displayed content.

An “addressable matrix” or “pixel matrix” is a transmissive element divided into pixels that can be individually (e.g., electrically) controlled as being “ON,” to transmit light, or “OFF,” to prevent light from passing, such that a light source passing through can modulated to create an image. The examples of displays above include such matrix elements. Generally, a “modulation matrix” is an element that is segmented such that light traveling incident on different portions of the modulation matrix experience different optical properties of the modulation matrix, the different optical properties being controllable. Such a layer is used to imprint spatial information, such as an image, onto the light. A modulation matrix may be absorptive, reflective, transmissive, or emissive; and it may comprise electrophoretic, absorptive, fluorescent or phosphorescent, mechanical, birefringent, electrooptic materials. An addressable matrix is an example of a modulation matrix layer. In some embodiments the optical properties of each portion of a modulation matrix depend also on the incident light (e.g., for a photochromic-based modulation matrix).

As used herein, the “display aperture,” “exit aperture,” and the like is the surface where the light exits the display system toward the exit pupil of the display system. The aperture is a physical surface, whereas the exit pupil is an imaginary surface that may or may not be superimposed on the aperture. After the exit pupil, the light enters the outside world.

As used herein, the “imaging aperture” is the area or surface where the light enters an imaging system after the entrance pupil of the imaging system and propagates toward the sensor. The entrance pupil is an imaginary surface or plane where the light first enters the imaging system.

“Image aperture optic,” “aperture optic,” “exit aperture optics,” and the like correspond interchangeably to a set of optical elements located at the display aperture surface. In some embodiments, the set contains only one element, such as a transparent window. Exit aperture optics protect the inside of the display system from external contaminants. Exit aperture optics are also used to prevent unwanted light from entering the display system. In a display system, “stray light” is unwanted light that interacts with the display system and travels along a substantially similar path as the desired image into a viewer's eyes. E.g., stray light includes ambient light that enters the system through an undesired entrance and finally exits through the display aperture to be visible by an observer, thus degrading the viewing experience. With exit aperture optics, such stray light prevents or mitigates this degradation by removing stray light or its effects. In some embodiments, exit aperture optics includes a wave plate and a polarizer. In some embodiments, it includes an anti-reflection coating. In the context of stray light mitigation, an exit aperture may also be called an “ambient light suppressor.”

In display systems that use ambient or environmental light as the light source, or in the systems disclosed herein, the light source is external to the other components of the display system, i.e., external to the housing that contains the light-guiding subsystem, light enters the display system through a set of optics called “entrance aperture optics,” “entrance optic,” “entrance aperture optic,” and the like In some embodiments, this set contains only one element, which may be a single transparent element to transmit the ambient light into the display system. Entrance aperture optics is located at the surface where the ambient light enters the display system. This surface is called the “entrance aperture,” “light entrance,” and the like. In some embodiments, the entrance aperture optics is configured to collect as much light as possible and may include diffractive optic elements, Fresnel lens or surfaces, nanocone or nanopillar arrays, antireflection layers, and the like.

The terms “field evolving cavity” or “FEC” refer to a non-resonant (e.g., unstable) cavity, comprising reflectors or semi-reflectors, that allows light to travel back and forth between those reflectors or semi-reflectors to evolve the shape of the wavefront, and consequently the monocular depth, associated with the light in a physical space. One example of an FEC may comprise two or more half-mirrors or semi-transparent mirrors, facing each other and separated by an air gap or dielectric of distance d. The light that travels from the first half-mirror, reflected by the second half-mirror, reflected by the first half-mirror, and finally transmitted by the second half-mirror will have traveled a total distance of 2d, which is the monocular depth. Thus, the monocular depth is larger than the length of the FEC. If, for example, the source of light is a pixel, which is approximately a point source, the FEC causes the spherical wavefront of the pixel to be flatter than it would be if the light traveled once through the gap.

In some embodiments, an FEC may be parallel to or optically coupled to a display or entrance aperture optics (in the case of display systems that use ambient light as the light source) or to an imaging aperture or imaging aperture (in the case of imaging systems). In some embodiments, an FEC changes the apparent depth of a display or of a section of the display.

As another non-limiting example, an FEC comprises a reflector and a semi-reflector oriented at an angle to the reflector. The semi-reflector receives and reflects light from a light source and directs it toward the reflector. The reflector receives said light, then reflects it toward the semi-reflector, which (partially) transmits the light to the outside world, towards a viewer. In an FEC, a round trip occurs once the light completes one cycle and comes back to the first (semi-)reflective component.

In some embodiments, a “round trip” occurs when light substantially reverses direction to interact with an element of an optical system more than once. The term “round trips” denotes the number of times that light circulates or bounces back and forth between two cavity elements, or the number of times light interacts with a single element.

FECs can have infinitely many different architectures, but the principle is always the same. An FEC is an optical architecture that creates multiple paths for the light to travel, either by forcing the light to make multiple round trips or by forcing the light from different sections of the same display (e.g., a segmented display) to travel different distances before the light exits the cavity. If the light exits the cavity perpendicular to the angle it has entered the cavity, the FEC is referred to as an off-axis FEC or a “FEC with perpendicular emission.”

An FEC assists in providing depth cues for three-dimensional perception for a user. In some embodiments, a depth cue is a monocular depth cue. The number of round trips is arbitrarily engineered. For example, there may be 0, 1, 2, or 3 round trips. The number of round trips substantially determines the monocular depth perceived be a viewer. In some embodiments, a monocular depth is larger than the distance between the viewer and the light source. For example, the ratio between the monocular depth and the distance may be 1, 1.1, 1.5, 2, 2.5, 3, 4.5, or 5. In some embodiments, the ratio may lie within a range, such as between 1 and 2, between 1 and 4, between 2 and 4, or greater than 2. In some embodiments, a monocular depth is dynamically adjustable by modifying a property of the virtual display system.

In some embodiments, polarization-dependent and polarization impact elements—such as polarizers, wave plates, and polarizing beam splitters—may be used to increase the light efficiency or modify the number of round trips. In some embodiments, different light rays travel different total distances to produce multiple focal planes, or a multi-focal image, which has a plurality of image depths. In some embodiments, an image depth is dynamic or tunable via, e.g., electro-optic structures that modify the number of round trips.

The “light efficiency” or “optical efficiency” is the ratio of the light energy the reaches the viewer to the light energy emitted by an initial display.

Throughout this disclosure, “angular profiling” is the engineering of light rays to travel in specified directions. Angular profiling may be achieved by directional films, holographic optical elements (HOEs), diffractive optical elements (DOEs), lenses, lenslet arrays, microlens arrays, aperture arrays, optical phase masks or amplitude masks, digital mirror devices (DMDs), spatial light modulators (SLMs), metasurfaces, diffraction gratings, interferometric films, privacy films, or other methods. “Intensity profiling” is the engineering of light rays to have specified values of brightness. It may be achieved by absorptive or reflective polarizers, absorptive coatings, gradient coatings, or other methods. The color or “wavelength profiling” is the engineering of light rays to have specified colors, or wavelengths. It may be achieved by color filters, absorptive notch filters, interference thin films, or other methods. “Polarization profiling” is the engineering of light rays to have specified polarizations. It might be achieved by metasurfaces with metallic or dielectric materials, micro-or nanostructures, wire grids or other reflective polarizers, absorptive polarizers, quarter-wave plates, half-wave plates, 1/x waveplates, or other nonlinear crystals with an anisotropy, or spatially profiled waveplates. All such components can be arbitrarily engineered to deliver the desired profile.

“Distortion compensation” is a technique for compensating errors in an optical system that would otherwise degrade image quality. In some embodiments, the distortion compensation is computational. The desired image content is pre-distorted such that when it experiences a physical distortion, the effect is negated, and the result is a clear image. Distortions to compensate include aberrations, angular variations of reflections. For example, a birefringent or anisotropic element may be added to account for an angle-dependent response of a wave plate. Such elements are called compensators or C-plates. Distortion compensation may also be affected computationally. For example, if a virtual display system produces a barrel distortion, a pre-computed image may include a pincushion-type distortion, such that the net effect is an image with minimal or zero barrel or pincushion distortion. Another type of distortion correction is perspective distortion correction. Other types of distortion compensation include perspective distortion compensation, which pre-compensates skewed based on off-axis reflections of optical elements. This can be pre-compensated using a homography transformation, keystone correction, and the like.

For example, the virtual image may have a barrel distortion that is produced by the nonuniform magnification of different elements of the image as they travel through a field-evolving cavity. The barrel distortion may be modeled as a function that transforms the image according to a polynomial function, such as f(r)=r(1−kr²), where r is the radial distance from the center of the image, and k is a system parameter. To pre-compensate this barrel, distortion, the inverse function g may be applied to the display content itself, where g(r)=r/(1−kr²). To apply this to the image, an algorithm may determine the pixel size of the display content, calculate the center pixel, create a matrix of the same pixel size of the image, and use g(r) to map each pixel value of the original display content to an element in the matrix. The radial distance is calculated by calculating the pixel distance between the pixel to be mapped and the center pixel. When all the pixels have been mapped, the matrix then becomes the new display content that is pre-compensated. The actual functions f and g depend on the specific configuration and shapes of the optics elements in the display system. Other types of compensation algorithms may use an inverse function, look-up table, machine learning algorithm, or neural network. In some embodiments, the pre-compensation may affect the intensities of the pixels or the color profile.

All such components and software can be arbitrarily engineered to deliver the desired profile. As used herein, “arbitrary optical parameter variation” refers to variations, changes, modulations, programing, and/or control of parameters, which can be one or a collection of the following variations: bandwidth, channel capacity, brightness, focal plane depth, parallax, permission level, sensor or camera sensitivity, frequency range, polarization, data rate, geometry or orientation, sequence or timing arrangement, runtime, or other physical or computational properties. Further parameters include optical zoom change, aperture size or brightness variation, focus variation, aberration variation, focal length variation, time-of-flight or phase variation (in the case of an imaging system with a time-sensitive or phase-sensitive imaging sensor), color or spectral variation (in the case of a spectrum-sensitive sensor), angular variation of the captured image, variation in depth of field, variation of depth of focus, variation of coma, or variation of stereopsis baseline (in the case of stereoscopic acquisition).

The “optic axis” or “optical axis” of a display (imaging) system is an imaginary line between the light source and the viewer (sensor) that is perpendicular to the surface of the aperture or image plane. It corresponds to the path of least geometric deviation of a light ray.

A “thin film” is a subwavelength-thick film or layer. Multilayer films comprise multiple thin films. Some films may be birefringent. In some embodiments, one or more layers are switchable, such as an LC thin film. Thin films and multilayer films may be coated onto solid substrates or other optical components.

A “programming element,” layer, or material is one whose properties can be transversely patterned or modified by a source such that when the source is incident on it locally, its properties change at that local position. Programmable layers are distinct from elements whose property change is global, i.e., occurs across the entire, or most of the element in a uniform way. For example, a polarizer, wave plate, semi-reflector, and the like are not light modulation layers because their responses are intended to be fixed. On the other hand, when one of those elements has higher-order properties, such as an optical nonlinearity, then the interaction may convert it into a programming element. Effectively, the programming element generates a transverse pattern that is coupled to a “modulation element,” layer, or material. Which is an element that receives the pattern of the programming element and transfers it to image-forming light. The nature of the coupling is arbitrary. For example, it may be optically coupled, thermally coupled, magnetically coupled, electrically coupled, mechanically coupled, and the like. In some embodiments, the programming and modulation elements are one in the same. This may be the case when the programming material is doped within a substrate of the modulating material, or when a single material is both programmable by a source and able to imprint a pattern onto image-forming light.

The terms “meshless optic, “meshless mask,” and the like refer to the combination of a programming element and modulation elements or materials that imprint a pattern onto image-forming light without an addressable matrix. In some embodiments, the term includes the secondary source that produces the pattern. The meshless optic is the subsystem that modifies a transversely varying optical property of light, particularly of image-forming light. The lateral size of the image is determined at least in part by the lateral sizes of the components of the meshless optic. In some embodiments, a lateral image size is greater than 5 cm, greater than 10 cm, greater than 15 cm, or greater than 20 cm.

“Diffractive artifacts” are artifacts that are caused by pixelated structures or addressable matrices. These include image pixelation, rainbow effects, diffracted waves at different grating orders, and the like. Generally, they distort the ideal image and are unwanted. A meshless optic serves to imprint a pattern onto image-forming light without introducing diffractive artifacts. Such diffractive artifacts are farfield diffractive artifacts and would be viewed by a viewer of a display system.

As used herein, “imaging system” refers to any apparatus that captures an image, which is a matrix of information about light intensity, phase, temporal character, spectral character, polarization, entanglement, or other properties used in any application or framework. Imaging systems include cellphone cameras, industrial cameras, photography or videography cameras, microscopes, telescopes, spectrometers, time-of-flight cameras, ultrafast cameras, thermal cameras, or any other type of imaging system. In some embodiments, the gesture that is output can be used to execute a command in a computer system connected, wireless or by hardwire, to the gesture camera.

Some capabilities described herein may be implemented in one or more modules. A module comprises the hardware and/or software, to implement the capability. For example, such a capability may be implemented through a module having one or more processors executing computer code stored on one or more non-transitory computer-readable storage medium. In some embodiments, a capability is implemented at least in part through a module having dedicated hardware (e.g., an ASIC, an FPGA). In some embodiments modules may share components. For example, a first function module and a second function module may both utilize a common processor (e.g., through time-share or multithreading) or have computer executable code stored on a common computer storage medium (e.g., at different memory locations).

In some instances, a module may be identified as a hardware module or a software module. A hardware module includes or shares the hardware for implementing the capability of the module. A hardware module may include software, that is, it may include a software module. A software module comprises information that may be stored, for example, on a non-transitory computer-readable storage medium. In some embodiments, the information may comprise instructions executable by one or more processors. In some embodiments, the information may be used at least in part to configure hardware such as an FPGA. In some embodiments, the information for implementing capabilities such as functions, visual templates, graphical user interfaces, input stream reception, and input stream generation may be recorded as a software module. The capability may be implemented, for example, by reading the software module from a storage medium and executing it with one or more processors, or by reading the software module from a storage medium and using the information to configure hardware.

A “medical diagnostic tool” is a system with which a patient interacts for medical imaging or diagnostic purposes, and may also be called “medical imaging tools,” “medical imagers,” “diagnostic imagers,” and the like. Medical diagnostic tools may be medical imaging systems such as magnetic resonance imaging (MRI) machines, functional MRI (fMRI) machines, magnetic resonance angiography (MRA) machines, X-ray machines, positron emission tomography (PET) scanners and other nuclear-based imagers, computed tomography (CT) imagers, mammography imagers, ultrasound imagers of any dimension, doppler-based ultrasound, optical imagers such as endoscopic or optical coherence tomography (OCT) systems, and the like. The invention disclosed here is useful for patients who may experience claustrophobia, vasovagal syncope responses, anxiety, stress, or other reactions when undergoing medical examination with a medical diagnostic tool. In some embodiments, the display system is integrated into non-medical tools

Each of the processes, methods, and algorithms described in the preceding sections may be embodied in, and fully or partially automated by, code components executed by one or more computer systems or computer processors comprising computer hardware. The processes and algorithms may be implemented partially or wholly in application-specific circuitry. The various features and processes described above may be used independently of one another or may be combined in numerous ways. Different combinations and sub-combinations are intended to fall within the scope of this disclosure, and certain method or process blocks may be omitted in some implementations. Additionally, unless the context dictates otherwise, the methods and processes described herein are also not limited to any sequence, and the blocks or states relating thereto can be performed in other sequences that are appropriate, or may be performed in parallel, or in some other manner. Blocks or states may be added to or removed from the disclosed example embodiments. The performance of certain operations or processes may be distributed among computer systems or computers processors, not only residing within a single machine but deployed across several machines.

This disclosure extends previous methods display systems which produce a single, continuous lightfield that enables simultaneous detection of monocular depth by each eye of a viewer who is positioned within the intended viewing region, where both the monocular depth can be greater than the physical distance between the display and the viewer, and where the apparent size of the display (as perceived by the viewer) is larger or smaller than the physical size of the display.

The methods in this disclosure can be used in arbitrarily engineered displays. These include, but are not limited to, large-scale lightfield displays that doesn't require glasses, systems that do require glasses, display systems that curve in front of the face and are closer to the user, lightfield displays with fractional lightfield, any type of head-mounted displays such as AR displays, mixed reality (MR) displays, VR displays, and both monocular and multifocal displays.

Further, the methods in this disclosure can be used in arbitrarily engineered imaging systems, including, but not limited to, microscopes, endoscopes, hyperspectral imaging systems, time-of-flight imaging systems, telescopes, remote imaging systems, scientific imaging systems, spectrometers, and satellite imagery cameras.

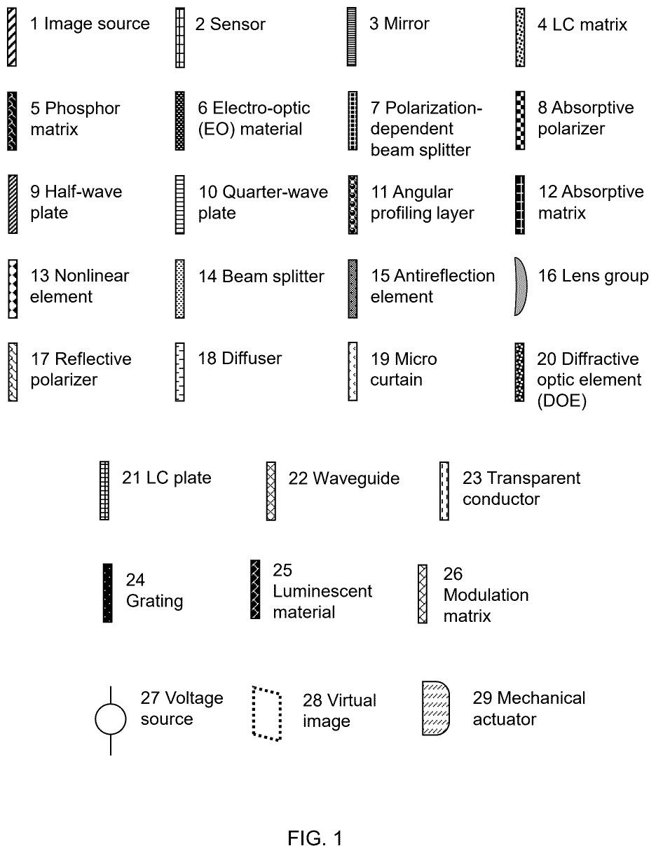

FIG. 1 depicts a set of elements that represent the fundamental components and structures of the embodiments disclosed herein.

An image source 1 (or light source) is any component or structure that emits light. In some embodiments, the light source generates an image. In some embodiments, the emitted light is an optical system to impact another component or structure. The light source may be one or more lasers, one or more light emitting diodes (LEDs), a backlight, a display panel, and the like. The intensity, polarization, luminance, angular profile, and spectrum can be arbitrarily engineered. In some embodiments, its properties change during its operation. For example, a laser beam scanner is a light source whose beam direction changes in time.

A display is a light source that produces an image. In this disclosure, the term “display” can be based on any technology, including, but not limited to, display panels likes liquid crystal displays (LCD), thin-film transistor (TFT), light emitting diode (LED), organic light emitting diode arrays (OLED), active matrix organic light emitting diode (AMOLED), micro LED, plastic organic light emitting diode (POLED), micro organic light emitting diode (MOLED), or projection or angular-projection arrays on flat screens or angle-dependent diffusive screens or any other display technology and/or mirrors and/or half-mirrors and/or switchable mirrors or liquid crystal sheets arranged and assembled in such a way as to exit bundles of light with a divergence apex at different depths or one depth from the core plane or waveguide-based displays. The display may be an autostereoscopic display that provides stereoscopic depth with or without glasses. It might be curved, flat, or bent; or comprise an array of smaller displays tiled together in an arbitrary configuration. The display may be a near-eye display for a headset, a near-head display, or far-standing display.

The spectrum of a display is arbitrary. For conventional images or virtual display systems, the display panels usually emit white light, which contains enough spectral components (e.g., red, blue, and green) such that the image is perceived as a white-light image or a full-color image.

A “segmented display” is a display in which different portions of the display show different display contents, i.e., a first portion of light from the segmented display corresponds to an independent display content compared to a second portion of light from the segmented display. In some embodiments, the light corresponding to each display content travels a different path through an optical system to produce correspondingly different virtual images. The virtual images may be at different monocular depths. Each display content is called a “segment.” In some embodiments, the different segments show identical content that are made to overlap to enhance brightness or another property of the image quality.

A “display system” is any device that produces images. Physical sources of display images can be standard 2D images or video, as produced by a display panel or a plurality of display panels. Such display technologies, or a plurality of them, may also be incorporated into other display systems. In some embodiments, spatial light modulators (SLMs) are used. In some display systems, light sources may be coupled with masks or patterned elements to make the light source segmented and addressable. Other sources may be generic light sources, such as one or several LEDs, backlights, or laser beams, configured for use, for example, in projection-based display systems. A display system may be a headset, a handheld device, or a free-standing system, where the term “free-standing” means that the device housing can rest on a structure, such as a table. In some embodiments, the display system is configured to be attached to a structure by a mechanical arm.

A sensor 2 is any transducer, array of transducers, or plurality of transducers that accepts information from the outside world and feeds that information into an apparatus. Sensors can be passive or active. Active sensors emit a signal, detect the return signal, and provide information based on differentiating features between the emission and the return. For example, an ultrasonic sensor, or generic time of flight sensor, calculates the distance to an object. In some embodiments, a sensor is an audio sensor such as a microphone. In some embodiments, the microphone is an optical microphone. In some embodiments, the sensor is an optical sensor that detects variations in ambient light intensity.

User input devices such as keyboard, mouse, and the like are sensors for purpose of this disclosure. A sensor also includes any type of camera, camera system, or camera array. For example, a camera may be a standard grayscale or color, e.g., RBG, camera, an infrared camera, a depth camera, a stereo camera, and the like.

In some embodiments a sensor or a plurality of sensors detects the geometry of a human, including the viewer of the display system, who may be the patient of a medical diagnostic tool. This includes, for example, detecting an eye gaze, eye position or orientation, or head position orientation. The resulting geometric information may be used to modify the image content. For example, in a display system that produces stereoscopic effects, the image may be based on the left and right eye position to maintain stereoscopic depth cues.

A mirror 3 is a specular reflector that reflects light with high reflectivity. Mirrors may be curved, flat, or free-formed to an arbitrary geometry shape. A mirror may alternatively be called a “reflector.” In some embodiments, the reflectivity of the mirror is due to a surface effect. In some embodiments, the reflectivity is due to a bulk effect or to the joint effect of multilayer films. For example, a dielectric stack of thin films functions as a mirror in some embodiments.

A liquid crystal (LC) matrix 4 is an addressable matrix comprising an array of electrically addressable LC cells, or pixels. The pixels of the of the LC matrix modulate the polarization of the incident light, such that a subsequent polarizer converts the polarization changes to intensity changes to produce an image.

A phosphor matrix 5 comprises at least one layer of phosphor material. In some embodiments, the phosphor materials are those used in current OLED devices. Some display devices are hybrid devices that combine fluorescent (dmac-dps, dmac-dmt for blue light) and phosphorescence (for red/yellow light). Some OLEDs use thermally active delated fluorescence.

Typically, phosphor materials are organometallic doped with iridium, platinum, or titanium. For example, Ir(ppy)3 contains iridium as the central metal atom and emits green light. Ir(piq)2(acac) is an iridium-based phosphorescent emitter, which emits deep blue light. Ir(MDQ)2(acac) is a blue-emitting phosphorescent material based on iridium. PtOEP: Platinum octaethylporphyrin is a phosphorescent material known for emitting red light. Ir(2-phq)3 is an iridium-based phosphorescent emitter that emits yellow light. FIrpic: is a blue-emitting phosphorescent material based on iridium and fluorine. PmIr is a phosphorescent material that emits blue light, composed of polymers with incorporated iridium complexes. PFO-DBTO2 is a blue-emitting phosphorescent material based on polyfluorene. Btp2Ir(acac) is a green-emitting phosphorescent material based on iridium. Ir(ppy)2(acac) is a green-emitting phosphorescent material containing iridium. DPVBi is an efficient blue phosphorescent emitter that is used to produce blue OLEDs. The yellow phosphorescent emitter is Ir(tptpy)2(acac).

Other phosphorescent materials use phosphorescent pigments that contain compounds like strontium aluminate, which is doped with rare earth elements like europium or dysprosium, for use in highlighters, emergency signs and markings. Some glow-in-the-dark paints or dial indicators contain phosphorescent pigments based on zinc sulfide or strontium aluminate. Luminous elements on some watch and clock dials may consist of phosphorescent materials like tritium-based paints (though tritium is radioactive) or non-radioactive compounds like strontium aluminate.

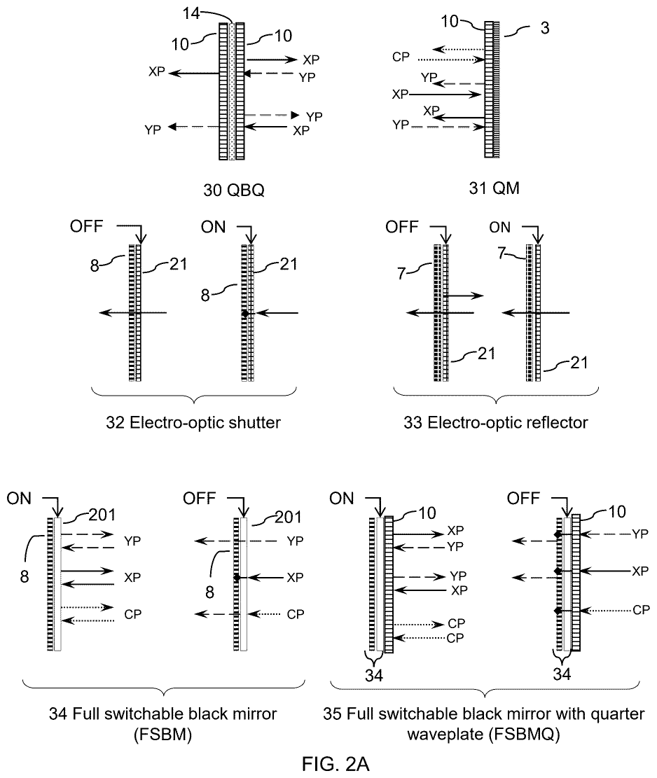

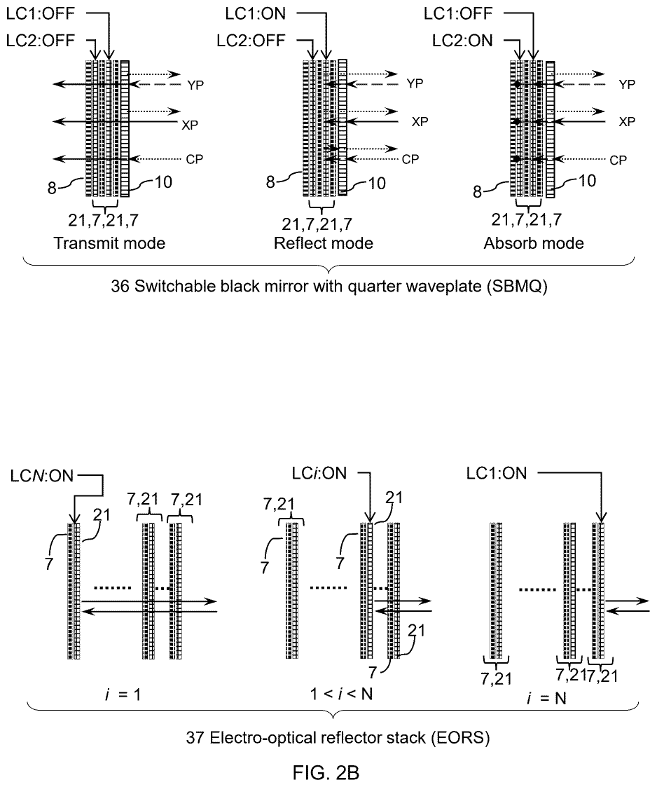

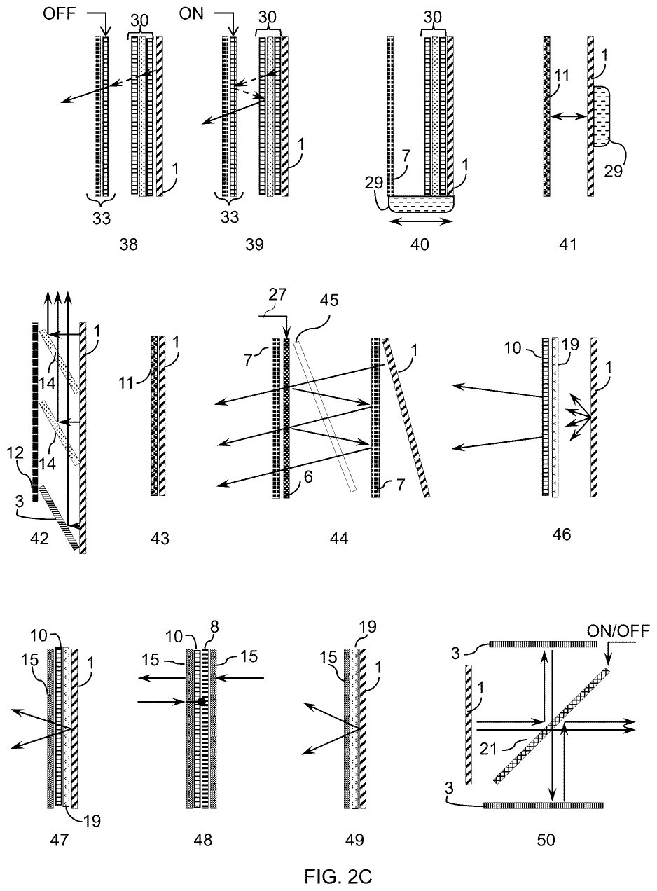

An electro-optic (EO) material 6 is a material whose refractive index changes with the application of an electric field. It is an example of a nonlinear element because the electric field may be caused incident light, which can experience that index change or cause a different light source to experience it. A photorefractive material is an example of an electro-optic material. When the field is caused by an external applied voltage, it is an active element. Throughout this disclosure, the terms “active design,” “active components,” or, generally, “active” refer to a design or a component that has variable optical properties that can be changed with an optical, electrical, magnetic, or acoustic signal. Electro-optical (EO) materials include liquid crystals (LC); liquid crystal as variable retarder (LCVR); or piezoelectric materials/layers exhibiting Pockel's effects (also known as electro-optical refractive index variation), such as lithium niobate (LiNbO₃), lithium tantalate (LiTaO₃), potassium titanyl phosphate (KTP), strontium barium niobate (SBN), and β-barium borate (BBO), with transparent electrodes on both sides to introduce electric fields to change the refractive index. The EO material can be arbitrarily engineered. Conversely, “passive designs” or “passive components” refer to designs that do not have any active component other than the display. EO materials include the EO-based subassemblies in FIGS. 2A and 2B. An EL material 6 may also be an EO rotator or an optically active material that rotates the polarization of the incident light by a specified amount.

A polarization-dependent beam splitter (PBS) 7 reflects light of one polarization and transmits light of the orthogonal polarization. A PBS can be arbitrarily engineered and made using reflective polymer stacks, nanowire grids, or thin-film technologies. Other PBSs include PBS cubes. In some embodiments, a PBS is interchangeable with a reflective polarizer.

An absorptive polarizer 8 transmits light polarized along its pass angle and absorbs cross polarized light.

A half-wave plate (HWP) 9 is a wave plate that produces a relative phase shift of 180 degrees between perpendicular polarization components that propagate through it. For linearly polarized light, the effect is to rotate the polarization direction by an amount equal to twice the angle between the initial polarization direction and the axis of the waveplate. In some embodiments, horizontally polarized light is converted to vertically polarized light, and vice versa, after transmission through an HWP.

A quarter-wave plate (QWP) 10 is a wave plate that produces a relative phase shift of 90 degrees. It transforms linearly polarized light into circularly polarized light, and it transforms circularly polarized light into linearly polarized light.

An angular profiling layer 11 is an arbitrarily engineered layer to produce a specified angular distribution of light rays. In some embodiments, it allows the transmission of rays within a certain range of incident angles, whereas rays outside such a range of angles are blocked. In some embodiments an angular profiling layer is a directional film or layer. This element selectively transmits light rays that are oriented at angles within a specified angular range and blocks light rays directed outside that range. For example, the directional film may transmit light rays that are incident within a range from zero to 10 degrees, zero to 20 degrees, zero to 30 degrees, zero to 40 degrees, zero to 50 degrees, or zero to 60 degrees. In some embodiments, the directional film tilts the chief ray of the light source. The directional film does not provide optical (focusing) power. In some embodiments, the directional film transmits an angular range that does not start at zero degrees. The directional film may be placed after a display. Another angular profiling layer example is a lenslet array. The lenslet array may be used in conjunction with a directional film to help focus or collimate the light. The lenselt array may be a microlens array. Each lenselt may be approximately the size, or smaller, than a pixel of a display.

An absorbing layer 12 is a material or element that absorbs light. In some embodiments, it is a black paint or coating. In some embodiments, it is vantablack.

A nonlinear element 13 is a material whose optical response is modified or impacted by light. Photorefractive elements are nonlinear. The nonlinear material is sometimes defined by the form of the nonlinearity, for example, a temporal or spatial nonlinearity, a Kerr-type, saturable-type, or higher-order nonlinearity. Nonlinear elements may be of different phases (e.g., solid, liquid, gas, plasma, and the like). Nonlinearities include harmonic generation, sum or frequency generation, rectification, and the like.

A beam splitter 14 is a specular reflector that partially reflects and partially transmits incident light. The ratio of reflected light to transmitted light can be arbitrarily engineered. In some embodiments, the transmission-to-reflection ratio is 50:50. In some embodiments, the transmission-to-reflection ratio is 70:30. A beam splitter is a semi-reflective layer that reflects a certain desired percentage of the intensity and transmits the rest of the intensity. A simple example of a beam splitter is a glass plate with a semi-transparent silver coating or dielectric coating on it, such that it allows 50% of the light to pass through it and reflects the other 50%. The term “semi-reflector” is used interchangeably.

Generally, both mirrors and beam splitters are used to direct light along a proscribed path in a display system. Both rely on specular reflection because their surfaces are smooth on the order of a wavelength. The term “specular reflector” therefore refers to both mirrors and beam splitters. The main difference is only the relative amount of light that is reflected. For example, with a perfect mirror, all the light is reflected, whereas in a standard beam splitter, about half the light is reflected. Though, a beam splitter may be designed to reflect other fractions of the light such as, for example, about 25% or 75%. How much light is reflected, the reflectance, may also vary by wavelength or polarization.

Specular reflectors, reflectors or semi-reflectors, such as mirrors or beam splitters, when inside an MRI bore should be free of metallic materials and other materials that may interact with a magnetic field. There are multiple ways to make a reflective surface without using metal or using materials that do not have a magnetic response that would disrupt MRI use. Such a material is called “non-magnetic.” In some embodiments, such non-magnetic materials are non-ferrous metals. In some embodiments, other optical components such as exit and entrance optics are also non-magnetic.

For use with an MRI machine, optical components are non-magnetic. Examples of non-magnetic materials include glass, possibly with silica coating (silicon dioxide) for mechanical protection, plastics such as acrylic, polycarbonate, PVC, PET, carbon-fiber-based plastics, non-magnetic ceramic materials, silicon carbine, polydimethylsiloxane (PDMS), stretched polyester films like Mylar (R), or other polymer-type materials such as polyethylene terephthalate. Some optical elements comprise thin films or multilayer films, also called multilayer stacks. In some embodiments, reflective coating layers may include magnesium fluoride (MgF₂), tantalum pentoxide (Ta₂O₅), titanium dioxide (TiO₂), polymer materials, graphite, or other non-magnetic dielectrics. In some embodiments, a reflector is a transparent thin film that has etched (via laser means, chemical means, embossing means, and the like) to create microstructures for a DOE, holographic element, grating or waveguide structure, and the like. In some embodiments, the optical components have gelatin or other flexible materials, such as hydrogels or similar polymers.

Polarizers and other polarization-dependent elements like wave plates may also be non-magnetic; for example, a polarizing film may be polyvinyl alcohol (PET) and have dichroic molecules. In some embodiments, other dichroic materials are used. Wave plates may also be non-magnetic, comprising birefringent crystals, quartz, MgF₂, mica, plastic, various polymers, and the like. Some non-magnetic liquid crystals may be used.

In some embodiments, the non-magnetic materials are non-ferrous materials that do not interact with an MRI machine. Some of these materials are metallic but do not exhibit a response that precludes them from use around MRI machines. Some examples include types of titanium, aluminum, brass, copper, bronze, and aluminum bronze alloys. Such materials, however, may not be ideal if they interfere with the MRI response.

An antireflection (AR) element 15 eliminates reflections of light incident on its surface. A microstructure such as a nano-cone layer may be an AR element. In some embodiments an AR element is a thin-film coating.

A lens group 16, which consists of one or multiple lenses of arbitrary focal length, concavity, and orientation. In some embodiments, a lens group forms a real image on an imaging sensor.

A reflective polarizer 17 transmits light polarized along its pass angle and reflects cross polarized light. A wire grid polarizer (a reflective polarizer made with nano wires aligned in parallel) is an example. The reflectivity and transmittivity depends on the angle of the incident light. In some embodiments, a reflective polarizer may be substituted for a polarization-dependent be3aem slitter and vice versa.

A diffuser 18 scatters light in a random or semi-random way. A diffuser can be a micro-beaded element/array or have another microstructure. Diffusers may reflect scattered light or transmit scattered light. The angular profile of the light may be arbitrarily engineered. In some embodiments, light scattered by a diffuser follows a Lambertian profile. In some embodiments, the light scattered forms a narrower profile.

A micro-curtain 19 redirects light into specified directions or shields light from traveling in specified directions. A micro curtain can be made by embedding thin periodic absorptive layers in a polymer or glass substrate, or it can be made by fusing thin black coated glass and cutting cross-sectional slabs.

A diffractive optical element (DOE) 20 has a structure to produce diffractive effects. The DOE can be of any material and may be arbitrary engineered. In some embodiments, a DOE is a Fresnel lens.

An LC plate 21 is a uniform LC slab or thin film. In the “ON” state, the LC plate rotates the polarization of the light that passes through it. In the “OFF” state, the state of the light polarization is unchanged upon transmission through the layer. In some embodiments the LC is a nematic twisted crystal. In some embodiments, the LC plate is doped with other particles or elements, such as quantum dots, resonant nanoparticles, and the like. In some embodiments, the doped particles are fixed in place, such as conducting rods that extend from one side to the other. Such an architecture effectively provides conductivity to the LC and allows current to pass through it. In some embodiments the LCs are slightly conducting. In some embodiments, either the programming layer or the modulation layer comprises a photorefractive or other EO materials. In some embodiments, the LC is dye-doped LC (methyl-red), but this may be too slow for certain applications. The LC may be of any type: ferroelectric, twisted nematic, cholesteric, and the like. In some embodiments, the material is an LC-PR hybrid material. In some embodiments, the LC is doped randomly with nanospheres, subwavelength structures, or other particles.

The LC plate may be of any type: twisted nematic, cholesteric, ferroelectric, nematic, smectic, discotic, and the like. Its specific structure and orientational/geometric properties can be arbitrarily engineered to produce the desired electro-optic effect. In some embodiments, an LC plate comprises layers of individual LC plates stacked on top of each other.

A waveguide 22 is a structure to guide light along a direction. In some embodiments, a display is formed by optically coupling a light source, such as a backlight, to a waveguide. In some embodiments, the waveguide comprises multiple waveguides or is wavelength dependent.

A transparent conductor 23 is a material that has simultaneously high optical transparency and good electrical conductivity. In some embodiments, a transparent conductor is a semiconducting material, which may be doped. For example, indium tin oxide (ITO) is a transparent conductor. Other transparent conductors include graphene, silver or cupper nanowires, carbon nanotubes, MoO₃, aluminum-or gallium-doped zinc oxide, and boron-doped diamond. Note that a transparent conductor may also be a transparent semiconductor.

A grating 24 is a corrugated structure to scatter light into specific directions. The corrugated structure is typically on the order of the wavelength of light, e.g., between 400 nm and 1000 nm, such that diffraction effects cause the scattering. In some embodiments, gratings are periodic. In some embodiments, a grating is a surface grating etched onto a substrate to in-couple or out-couple light into or out of the substrate.

A luminescent material 25 emits light. In some embodiments, the luminescence is phosphorescence, or it is fluorescence, which are “photoluminescent” materials. Luminescent materials' light emission may be caused by the absorption of light, usually at a different wavelength. In some embodiments, there is IR to visible up conversion. In some embodiments, the fluorescent particles comprise quantum dots, such as CdS. In some embodiments the photoluminescent materials are activated, switched, or otherwise modified by another light source. A “quantum dot” (QD), or “quantum-dot layer,” is a fluorescent particle light source, or an element containing a plurality of such light sources, which are based on the absorption and emission of light from nanoparticles in which the emission process is dominated by quantum mechanical effects. These particles are a few nanometers in size, and they are often made of, but not limited to, II-IV semiconductor materials, such as cadmium sulfide (CdS), cadmium telluride (CdTe), indium arsenide (InAs), or indium phosphide (InP). When excited by ultraviolet light, an electron in the quantum dot is excited from its valence band to its conduction band and then re-emits light as it falls to the lower energy level. In some embodiments, QD spectra are modified by structure, morphology, temperature, strain.

Other luminescent materials or elements may be photoactivated or photoswitchable, which is activated to absorb light at a first wavelength and emit it at a second wavelength only in the presence of a third wavelength. Photoswitchable and photoactivated materials include fluorescent proteins such as PA-GFP, PAmKate, Denddra2, Kaede, EosFP, Dronpa, Kindling FP, and the like. The absorption, emission, and activation spectra can be arbitrarily engineered. Further examples include azobenzenes, spiropyrans, and diarylethenes, as well as donor-acceptor Stenhouse adducts, phototropic organic metals or metal oxides, some QDs, perovskites, and some ruthenium, iron, or cobalt complexes.

A modulation matrix 26 is an element that is segmented such that light incident on different portions of the modulation matrix experience different optical properties of the modulation matrix, the different optical properties being controllable. Such a layer is used to imprint spatial information, such as an image, onto the light. A modulation matrix may be absorptive, reflective, transmissive, or emissive; and it may comprise electrophoretic, absorptive, fluorescent, or phosphorescent, mechanical, birefringent, electrooptic materials. An addressable matrix is an example of a modulation matrix layer. In some embodiments the optical properties of each portion of a modulation matrix depend also on the incident light (e.g., for a photochromic-based modulation matrix).

In some embodiments, a modulation matrix example is a spatial light modulator (SLM), which spatially modulates the amplitude or phase of light incident on it. An SLM may operate in reflection mode or transmission made, and it may be electrically addressable or optically addressable. In some embodiments, an SLM is used as a modulated matrix. In some embodiments a digital micromirror device (DMD), which is an opto-electrical-mechanical mirror comprising mirror segments or pixels that each reflect light in a desired direction, serves as the modulation matrix. Light incident on pixels corresponding to an image are directed in one direction, and unwanted light is directed into another direction. A DMD may be a modulation matrix.

A voltage source 27 is a source of electric voltage. In some embodiments, it is a power supply, a battery, an alternating current (AC) signal, or an electronic signal.

A virtual image 28 is defined above as an image to be viewed by a viewer.

A mechanical actuator 29 physically moves the elements to which are connected via an electrical or other types of signals.

FIGS. 2A through 2C show how the basic elements in FIG. 1 can be combined to produce structures, elements, architectures, subassemblies, or sub-systems. In some embodiments, these are integrated into a single, monolithic element, e.g., when a substrate is coated with various films or coatings. In some embodiments, they may be discrete components arranged with or without air gaps between them. In FIG. 2A, a QBQ 30 comprises a QWP 10, a beam splitter 14, and another QWP 10. Light incident on a QBQ is partially reflected and partially transmitted, and the QBQ acts as a HWP for both the reflected and transmitted portions, converting x-polarized light (XP) into y-polarized light and vice versa. In some embodiments the beam splitter is a PBS. A QM 31 comprises a QWP 10 and a mirror 3. It reflects all light, and it converts x-polarized light into y-polarized light and vice versa (or, equivalently, horizontally polarized light into vertically polarized light). It does not change the polarization state of circularly polarized light.

An electro-optic shutter 32 comprises an LC plate 21 and an absorptive polarizer 8. When the LC plate is ON, it rotates the polarized incident light such that it is aligned perpendicular to the absorptive polarizer and is absorbed by it. When the LC plate is OFF, it leaves the polarization unchanged and parallel to the absorptive polarizer which transmits it. An electro-optic reflector 33 comprises an LC plate 21 and a PBS 7. When the LC plate is ON, it rotates the polarization such that it aligned along the transmit orientation of the PBS. When the LC layer is OFF, the light passing through it is aligned such that the PBS reflects it.

A fully switchable black mirror (FSBM) 34 comprises an absorptive polarizer 8 and a full switchable mirror 201, which may be an EO material. In the ON state, the full switchable mirror 201 is on and reflects light of all polarizations. In the OFF state, the switchable mirror transmits the light, and an absorptive polarizer 8 extinguishes x-polarized light, transmits y-polarized light, and transmits only the y-component of circularly polarized light. A full switchable black mirror with quarter waveplate (FSMBQ) 35 comprises an FSBM 34 and a QWP 10. In the ON state, it reflects all light and interchanges x-polarized with y-polarized light (and vice versa). It reflects circularly polarized light without changing the polarization. In the OFF state it extinguishes circularly polarized light, transmits y-polarized light, and coverts x-polarized light into y-polarized light and transmits the result.

Shown in FIG. 2B are two switchable reflective stacks. A switchable black mirror with quarter waveplate (SBMQ) 36 comprises a QWP 10, followed by two alternating layers of LC plates 21 and PBSs 7, and finally one absorptive polarizer 8. The difference between the FSBMQ and the SBMQ is their corresponding polarization dependence. In the former the total reflectivity of the material is changing, agnostic to the polarization of the incident light, whereas the latter element produced a polarization-dependent reflectivity.

For the SBMQ 36, when both LC plates are OFF (“transmit mode”), all incident polarizations transmit an x-polarized component; incident linear polarization reflect circular polarization. Incident circular polarization reflects light that depends on whether it is right-or left-circularly polarized. When the first LC plate is ON and the second OFF (reflect mode), all light is reflected as circularly polarized. When the plate LC plate is OFF and the second LC is ON (absorb mode), incident light that strikes the absorptive layer and is extinguished, and no light is transmitted through the layers.

An electro-optical reflector stack (EORS) 37 comprises a stack of N alternating PBS 7 and LC plates 21. All but one LC plate is in the OFF state, and the LC plate that is in the ON state reflects the incident x-polarized light. All other layers transmit light. By varying which LC layer is in the ON state, the EORS modulates the optical depth or optical path or the length that the light must travel through the stack before it is reflected by a cross-polarized PBS layer next to the ON LC layer. In some embodiments the LC plates and PBSs are configured to reflect y-polarized light.

It should be noted that the EO shutter 32, the EO reflector, the FSBM 34, the FSBMQ, the SBMQ36, and the EORS 37 are all electro-optic and therefore may be used as an electro-optic programming element or modulation element.

Shown in FIG. 2C are further combinations of elements. In some embodiments, these form a variety of field evolving cavities (FEC) or layer stacks that can be used as subsystems for architectures explained throughout the disclosure. 38 and 39 are OFF and ON states, respectively, of a display 1 and QBQ 30 followed by an electro-optic reflector 33. In the OFF state, the light directly exits the device to be viewed by an observer. In the ON state, the light is forced to travel one round trip in the cavity, and the displayed image appears to be deeper compared to the actual location of the display. In some embodiments, the monocular depth of the resulting image is approximately twice as far as that of the display itself. 40 is a display 1 followed by a QBQ 30 and a PBS 7 set on a mechanical actuator 29. The actuator shifts the set of layers to create longer or shorter optical path lengths for the light and hence shorter or longer monocular depths. 41 is a mechanical actuator 29 fixed to display 1. The actuator can shift the display relative to an angular profiling element 11 to force the light to change directionality or to become collimated. In some embodiments, the angular profiling layer is a lenslet array such that the mechanical movement of the display changes the object distance and therefore impacts the collimation. In some embodiments, the display is “macro-formed,” meaning it may have mechanical waves or bends induced onto it by the mechanical actuators so that the directionality or collimation of the light that comes out of the angular lenslet array is impacted in a desired way. In some embodiments other elements, such as a beam splitter or mirror, are macro-formed.

In some embodiments, the display is mechanically shifting, because of the actuator's motion along a translational axis, again to impact the directionality of the exit light from the apertures. The mechanical actuation mechanism may be arbitrarily engineered. In some embodiments, the mechanical actuator is an array of ultrasonic transducers; in some embodiments, the mechanical translation is performed by a high rotation-per-minute brushless motor; in some embodiments, the mechanical movements are delivered via a piezo-or stepper motor-based mechanism.

An example of one type of FEC 42 consists of display 1 that is partitioned into segments, i.e., a segmented display. Light from the bottom segment is reflected by a mirror 3, and light from the upper segments is reflected by subsequent beam splitters 14. An absorptive matrix12 absorbs unwanted stray light. In some embodiments the absorptive matrix is a uniform attenuator to substantially absorb all the light incident on it uniformly across its surface. This is an example of an off-axis FEC. In some embodiments, the FEC produces a multifocal image. The FEC can be arbitrarily engineered to represent the desired number of focal planes.

Precavity optics 43 consists of display 1 layer followed immediately by an angular profiling element 11, which may be a directional film here. The angular profiling layer might be a lenticular lens array to provide stereopsis to the viewer, or it might be a lenslet array or any other angular profiling layer to provide autostereoscopic 3D or provide different images to different angles.

In some embodiments, the precavity optics comprises different elements to achieve the desired profiling. Such modified precavity optics may have fewer or more components.

An example of a tilted FEC 44 is an angled display 1, followed by a FEC comprising an “internal polarization clock” whose ends are composed of PBSs 7. In between the PBSs 7 is an EO material 6 that acts as a polarization rotator and a birefringent element (which is a material whose refractive index depend on direction of travel and/or polarization, i.e., an anisotropic material) 45, such that different angles of propagation result in different phase retardation of polarization. Another EO material 6 acts as shutter element that uses an electronic signal 27 that turns the light into a desired polarization so that only one of the round trips are allowed to exit the cavity, and the transmitted light has traveled a desired optical path or depth. This is a representation of a coaxial FEC with polarization clocks and segmented gated apertures with desired gating mechanisms. In some embodiments, each of these elements is segmented, such that light from different portions of a segmented display travel different distances.

Assembly 46 is a display 1 followed by a micro-curtain 19 and a QWP 10 to function as pre-cavity optics. This allows desired profiling of the light of the display. The pre-cavity optics can adjust the polarization, angular distribution, or other properties of the light entering the cavity. 47 shows of a stack of elements: a display 1, a QWP 10, a micro-curtain layer 19, and an antireflection element 15. This subsystem is used in many disclosed systems and is categorized as a display. The micro curtain can be arbitrarily engineered, and it allows for control of the directionality of the light and the visibility of the display. The AR layer allows for reduction of ambient or internal reflections of the systems that use this subcomponent. In some embodiments, the AR element is a coating on substrate.

Postcavity optics 48 is a sub-assembly consisting of an AR element15 and an absorptive polarizer 8 on one side facing a viewer and outside world, and a QWP 10 another optional AR element 15 or film on the side that faces the display from which light exits. In some embodiments, the AR element is a coating on substrate. In this disclosure, 48 is an example of aperture optics called an ambient light suppressor. In some embodiments, the ambient light suppressor is the final set of optical elements that the light experiences before exiting the display system. In some embodiments, the ambient light suppressor further comprises a directional film or angular profiling layer to produce angular profiling of the light exiting the system.

Postcavity optics 48 functionally mitigates the nonuniformity (waviness) observed in the virtual image and decreases the ambient light noise received by the user. Some part of the ambient light reflects directly from the shield layer, and some part of the ambient light enters the cavity and comes back. In some embodiments, it is an aperture optic to transmit light from the display system to the outside world. It can be a stack of layers laminated or deposited together such that the light that enters the cavity changes polarization and is absorbed by the stack of polymers. In some embodiments, depending on the polarization of the signal light or the image light, it is tilted or bent to further decrease the ambient light and internal reflections of an FEC. In some embodiments, it is composed of absorptive polarizers 8, QWPs 10, or arbitrary antireflection coatings 15. In some embodiments, it has an absorptive layer 12 to further decrease the ambient reflection because the ambient light passes twice through the shield layer. In some embodiments, Postcavity optics 48 has a liquid crystal layer 21 or optically tunable layer such that the electric signal applied can be leveraged to choose the image depth that needs to exit the cavity. In some embodiments, there is a liquid crystal layer with oscillating polarization on the shield layer to provide both polarizations to the outside world.

Subassembly 49 is a subassembly of a display with micro curtain layer and an AR element 15 on top.

An example of an off-axis, or non-coaxial FEC 50 is a sub-assembly consisting of two mirrors 3 on the top and bottom, a display 1 at the back, and an angled PBS 7 with LC plate 21 in the middle such that an ON/OFF electronic signal to the LC can change the length that the light must travel before it exits the cavity. In some embodiments, a stack of such angled PBS-on-LC splitters such that the length of the light travel can be programmed or controlled in multiple steps. In some embodiments, the mirror is a QM to rotate the polarization of the light.

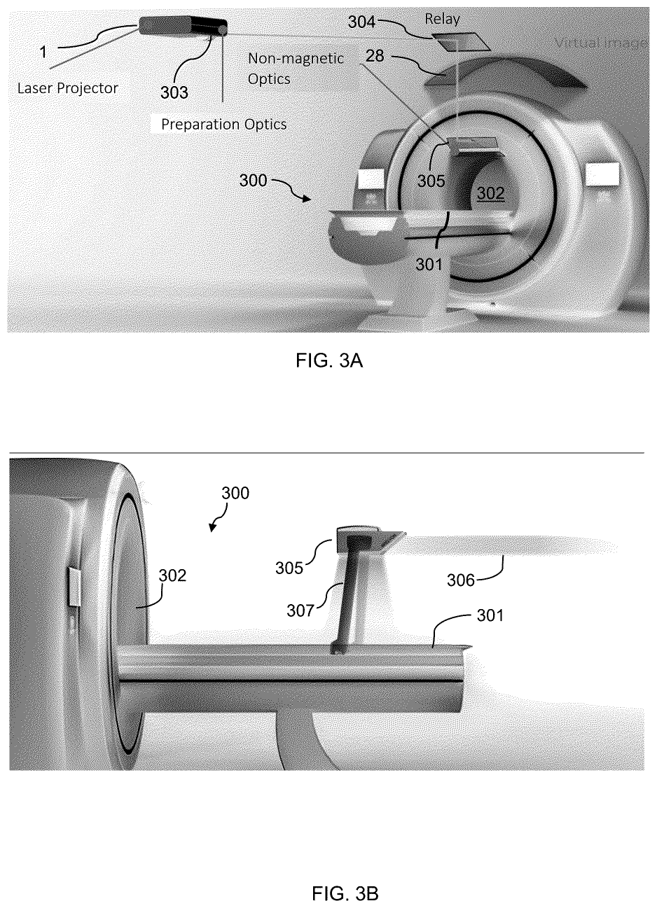

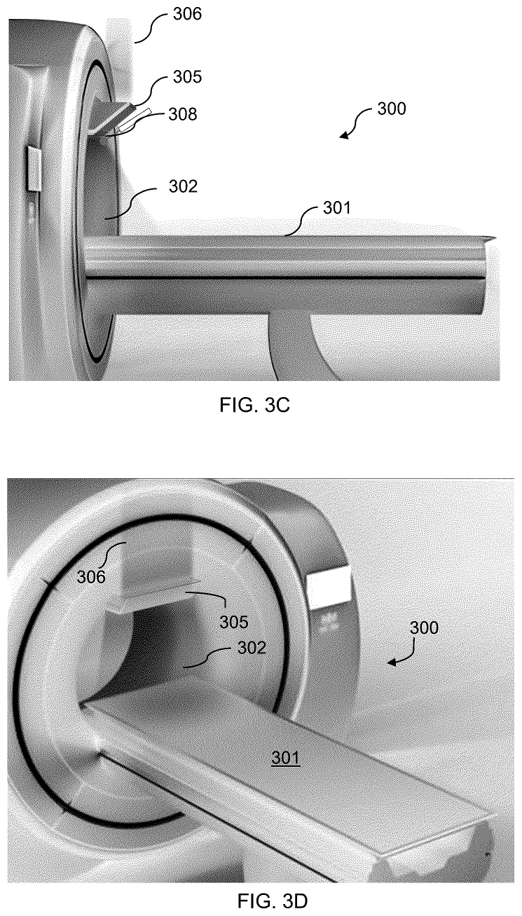

FIGS. 3A through 3D illustrate various embodiments of the invention disclosed. In such embodiments, the display system is integrated into or onto a medical diagnostic tool, including an MRI machine, with the image-forming light source exterior to the display system or to the medical diagnostic tool. The display system produces images that have depth cues, such as monocular depth cues or stereoscopic depth cues. Monocular depth cues are such that the viewer's eyes accommodate a distance farther than the radius of the cavity or bore of the medical diagnostic tool. Further, in some embodiments, the lateral dimensions of the virtual image are bigger than the light source dimensions or of the exit aperture or aperture optics placed at the exit aperture. With the display system producing such imagery, the viewer (the patient) experiences expansive imagery that mitigates claustrophobic effects.

In FIG. 3A, a medical diagnostic tool 300 includes a platform 301 on which a subject or patent rests. In some embodiments, the medical diagnostic tool is a magnetic resonance imaging (MRI) machine. In some embodiments, it is an X-ray, PET scanner, CT scanner, and the like. In some embodiments, the platform 300 supports an entire human body. In some embodiments, it is intended to support only a body part such as a limb. The platform is shaped such that the subject or patient is sitting upright or reclined or prone. The platform 301 is movable relative to the medical diagnostic tool and may enter a cavity 302 of the tool. In some embodiments, the cavity 301 is a bore of an in-bore MRI machine. In such an embodiment, the patent may enter feet first or head first. In FIG. 3A a patent may enter the cavity 301 feet first, and a display system 305 may be fixed to a surface of the medical diagnostic tool, such that the patent may view content from the display system while his lower extremities are in the cavity 301. In the context of this disclosure, the display system 305 does not necessarily include the image-forming light source. In some embodiments, the components and housing of the display system 305 are non-magnetic so as not to interfere with, or be damaged by, an MRI machine.

Image content is generated by a light source 1 such as a laser projector. The light that is generated passes through preparation optics 303 to profile the optical signal. The profiling is arbitrary and can including wavelength profiling, angular profiling, intensity profiling, or polarization profiling. In some embodiments, a relay 304 directs the light into the display system 305. The relay may be a specular reflector, such as a mirror or beam splitter. In some embodiments, the relay is a component of the display system itself. In some embodiments, the preparation optics 303 and the relay 304 are non-magnetic. The patient who is experiencing the display content when viewing the display system 305 will see a virtual image 28, which has a monocular depth that is farther from the viewer than is the display system itself. In some embodiments, the virtual image 28 lies in a focal plane. In some embodiments, the focal plane is curved.

FIG. 3B shows another embodiment of the invention in which the medical diagnostic tool 300 has a platform 301, and the display system 305 is attached to the platform via a mechanical support 307. Thus, the platform and the display system move together as they enter or exit from the cavity 302. Image-forming light 306 enters the display system from the side. In some embodiments, the virtual image is formed in a headbox below the display system 305, such a viewer is lying with his head on the platform below the display system. In some embodiments, the patient enters the cavity 302 head first.

FIG. 3C shows an embodiment in which the display system 305 is fixed to an exterior surface of a medical diagnostic tool 300 such that a viewer may light on the platform 301 and entire the cavity 302 feet first. In this embodiment, image-forming light 306 may enter the display system 305 from the top, and a viewer sees a virtual image when looking into an aperture 308 of the display system 305. FIG. 3D is an alternative perspective of the medical diagnostic tool 300 of FIGS. 3A and 3C. The image-forming light 306 enters the display system 305 from the top, and a patient or viewer lying on the platform 301 may enter the cavity 302 feet first. When the platform enters the cavity with the patent, the viewer's head lies in the headbox of the display system 305 to observe a virtual image.

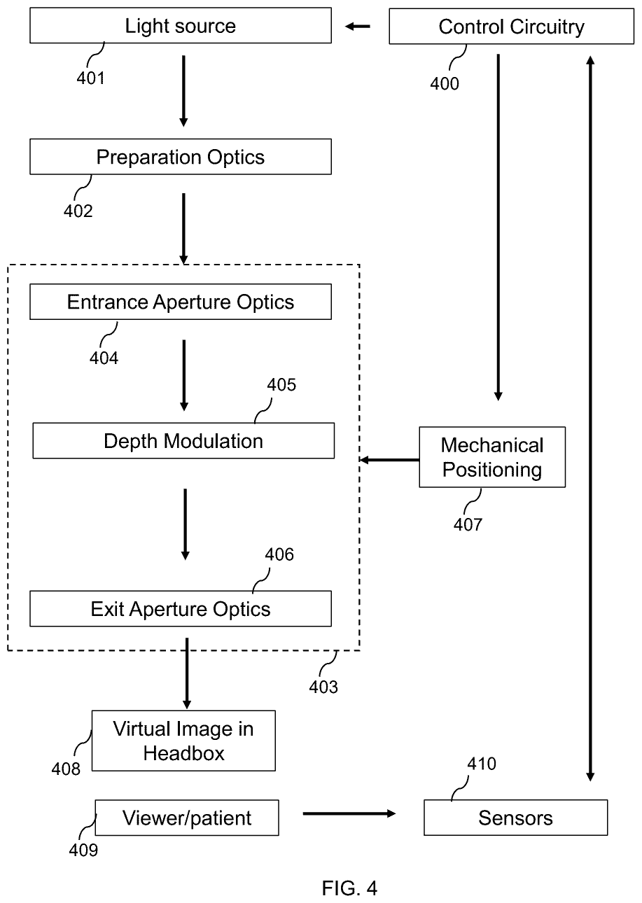

FIG. 4 shows a block diagram of the principal functions of the different structures of the invention. The first function that happens is that light is generated during the light source block 401. The light source can be a standalone image generator. It may be a display panel, a laser projector, or an LED or laser source coupled to a spatial light modulator, digital mirror device, and the like. The image-forming light is then profiled during the preparation optics block 402. For example, the preparation optics may include a polarizer or a wave plate to impact the polarization of the image forming light. In some embodiments, the preparation optics includes a diffusing element to smooth out intensity variations. In some embodiments, it includes an angular profiling layer such as a directional film. IN some embodiments, in includes a lens group or a lenslet array to collimate the image-forming light. In some embodiments, a curved reflector is a conjugate reflector and takes on the shape of a partial ellipse. In some embodiments, a semi-reflector during the depth modulation step is diffractive or nanostructured, or it is a metasurface.

After the light is prepared, it is acted upon during the display system block 403, which has separate subblocks. First, the light enters the system during the entrance aperture optics block 404. During this step, the light may be further prepared and profiled. In some embodiments, the light preparation happens at this block. In some embodiments, entrance aperture optics is a transparent window that protects the internal surfaces of the display system from exterior environmental effects, such as dust, or from physical manipulation. The light is modulated at the depth modulation block 405. During this step, the light may interact with a FEC to evolve its wavefront or equivalent. In some embodiments, the depth modulation effect varies across a lateral dimension of the image, such that different portions of the image obtain different depth values. In some embodiments, the image-forming light is partitioned into different segments such that each segment is depth modulated to a different depth. The segments may overlap to form a multifocal image. In some embodiments, the depth modulation block 405 includes a curved element, such as a curved mirror or refractive element to achieve or impact the depth modulation. In some embodiments, the depth modulation is variable, such that the image obtains different monocular depths at different times.

The light then is made to exit the display system during the exit aperture optics block 406. Any final optical profiling is achieved during this step. The exit aperture optics may comprise a transparent window. In some embodiments, the exit aperture optics includes elements to eliminate stray light, such as AR coatings/elements, absorptive polarizers, and wave plates. In some embodiments, the exit aperture optics serves to block the viewer from seeing ambient light that is incident on the exit aperture optics.

In some embodiments, an internal surface of the display system includes absorbing layers, black paint, matte textures, or other dark surfaces to help absorb unwanted stray light.

The light that exits then travels into a headbox to be viewed as a virtual image to an observer whose eyes are located within the headbox. This occurs at the virtual image in headbox step 408. The virtual image lies at a focal plane with a monocular depth. In some embodiments, the focal plane is curved. In some embodiments, the headbox is partitioned into a left eyebox and a right eyebox, such that a respective left virtual image and a right virtual image are viewed by a respective left eye and right eye of a viewer. In such embodiments, the viewer experiences both monocular and binocular depth cues. In some embodiments, a focal plane is curved such that it approximates a human horopter.

During viewing, the viewer/patient block 409 allows for feedback. The motion of the viewer, including the viewer's head or the eyes, as well as any other biometric data may be recorded or captured by sensors in a sensors block 410. In some embodiments, the sensors are non-magnetic. In some embodiments, the sensors are located outside a bore or cavity of a medical diagnostic tool and remotely capture such information. In some embodiments, the sensors are optical systems that use optical signals, such as infrared (IR) signals. IN some embodiments, the sensors are sensor arrays. In some embodiments, they capture a position of a viewer or his eyes and feed that into control circuitry during a control circuitry block 400. The control circuitry may use that information to adjust the image content or use a mechanical positioning block 407 to adjust a component or subsystem of the display system.

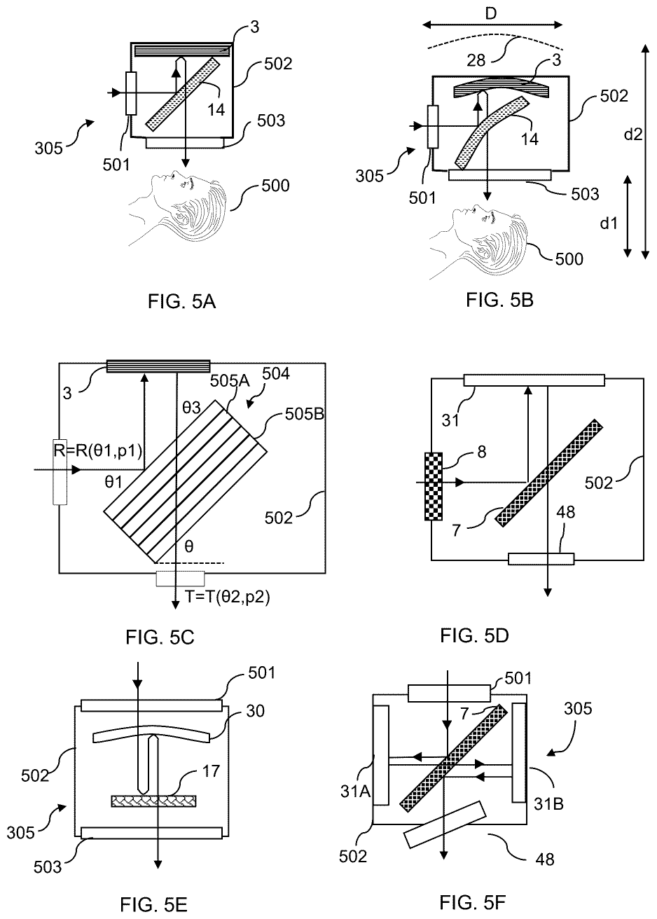

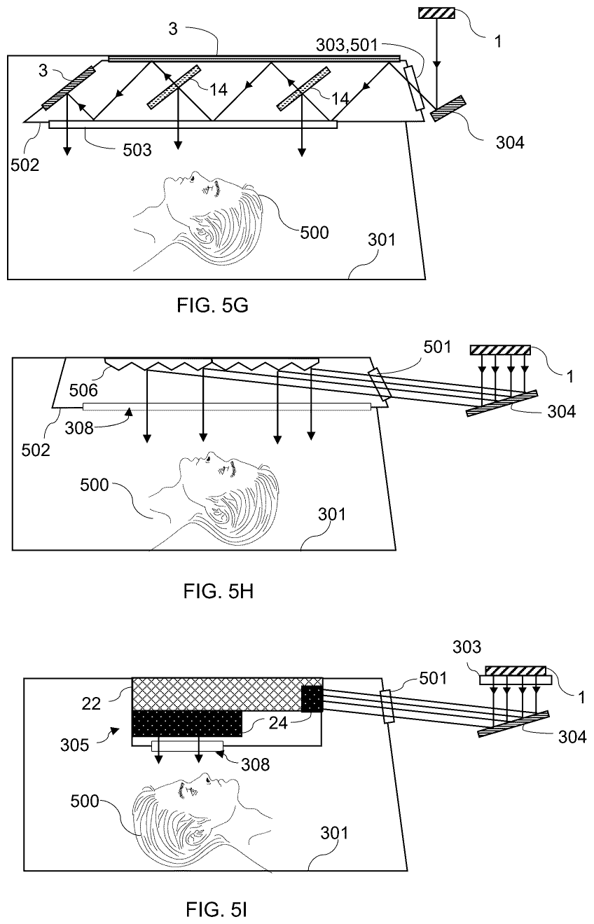

FIGS. 5A through 5H show various embodiments of the display system and how it delivers a virtual image to a viewer. In FIG. 5A, a viewer 500 is positioned to be within the headbox of a display system 305. Light enters the system through an entrance optic 501 (i.e., the entrance aperture optics) and is depth modulated by a pair of specular reflectors including a semi-reflector (beam splitter 14) and a reflector (mirror 3). In this embodiment, the light is incident on the semi-reflector twice. The light then exits the system through an exit optic 503 (i.e., the exit aperture optics) to be viewed by the viewer. The components of the display system are fixed to a housing 502 which itself may be secured to a mechanical support or directly to a surface of a medical diagnostic device. In this embodiment, in which specular reflectors are flat, the optical path length obtained within the system is equal to the physical distance traveled, here shown by a chief ray.

The entrance optic 501 may comprise a polarizer, directional film, absorptive polarizer, and/or antireflection coating and may be curved. The Exit optic 503 may comprise one or more antireflection elements, an absorptive polarizer, and a quarter wave plate, and may be curved. Both structures may be tilted to further impact the quality of the virtual image. The tilt may be at an oblique angle relative to a surface of the housing 502, i.e., to the surface where the light enters or exits. In some embodiments, the tilt angle is between 0 and 5 degrees, 4 and 10 degrees, 5 and 15 degrees, or 10 and 30 degrees.

In some embodiments, one or more of the specular reflectors are curved. For example, in FIG. 5B, light enters the display system 305 through an entrance optic 501, which may include profiling optics such as a directional film, polarizer, or wave plate, e.g., a half-wave plate or a quarter-wave plate. The light is reflected by a beam splitter 14, which is curved, and then reflected by a curved mirror 3, which reflects the light back through the beam splitter 14 through the exit optic 503 such that a viewer 500 sees a virtual image 28, which is curved. In some embodiments, a lateral dimension D of the virtual image is larger than the exit optic 503. In some embodiments, it is between 1.5 and 3 times as big as a lateral dimension of the exit optic. In some embodiments, it is more than twice as big, more than three times as big, or more than 4 times as big. In some embodiments, the virtual image 28 possesses a monocular depth d2 that is bigger than a distance d1 between the viewer 500 and the display system 305, e.g., between the viewer 500 and the exit optic 503. In some embodiments, in some embodiments, the monocular depth is between 1.5 and 3 times as big. In some embodiments, the monocular depth is more than twice as big, three times as big, four times as big, or five times as big as a distance between the viewer 500 and the display system. In some embodiments, the monocular depth of the virtual image 28 spans a range across its focal plane, ranging from 1 to 2 m, 1 m to 5 m, 1.2 to 2 m. 1.2 m to 3 m, 2 m to 5 m, or 1 m to 3 m.

In some embodiments, the distance d1 between the viewer 500 and the display system 305 is such that both the viewer and the display system fit inside a bore of a medical diagnostic tool such as an MRI. In some embodiments, this distance is less than 70 cm, less than 60 cm, 50 cm, 40 cm, 30 cm, 20 cm, or 10 cm.

Similarly a lateral dimension of the virtual image 28 may be bigger than a physical lateral dimension of the exit aperture optic 503. It may be greater than 1.1 times, greater than 1.5 times, greater than 2 times, greater than 2.5 times, greater than 3 times, or greater than 4 times. In some embodiments, it is between 1.1 and 5 times greater.

In some embodiments, the curvature of one or more of the specular elements is such that the monocular depth is brought closer to a viewer 500 compared to an optical path distance between a viewer and the light source, preparation optics, or entrance optics 501. Further, some of the specular reflectors, such as the mirror 3, may be a retroreflector to achieve a similar effect. In some embodiments, the curvature forms a conjugate mirror. In some embodiments, the curvature is a freeform surface.

In any embodiment, the optical components, subassemblies, structures, and mechanical elements and housing must comprise materials and be of the correct geometry so as not to interfere with the function of the medical diagnostic tool to which it mounted; to injure a viewer, patient, technician, or medical professional; damage the tool or a part of it; or affect any imaging signaling or electronics. For example, if the medical diagnostic tool is an MRI machine, the various components should be non-magnetic. In some embodiments, this includes any preparation optics or relay exterior to the housing of the display system or exterior to the MRI machine.

Similar to the discussion above, there are multiple materials to make non-magnetic specular reflectors, such as mirrors, semi-reflectors like beam splitters, various entrance and exit aperture optics, polarization-dependent elements, and the like.

Please note that some of these methods may require specialized equipment or expertise, and the quality of the mirror may vary depending on the materials and techniques used.

For example, specular reflectors may be made of a stack of dielectric thin films. As shown in FIG. 5C, the FEC comprises a dielectric stack 504, which serves as a semi-reflector to receive the light, reflect toward a mirror 3, the mirror subsequently reflecting the light back toward the dielectric stack 504, which then transmits the light to the outside world. The mirror 3 and dielectric stack 504 are mounted to the housing 502, which has an entrance aperture and exit aperture to respectively receive the light from a light source and transmit the light to a viewer. In some embodiments, at the apertures are located exit or entrance aperture optics as described previously, e.g., in FIGS. 5A and 5B. The dielectric stack comprises a set of thin films, including a first thin firm 505A and a second thin film 505B. The thickness of each of the thin films is usually less than a wavelength, for example, a quarter of an optical wavelength, a half of an optical wavelength, and the like. In some embodiments, the thin films are periodically arranged in a repeating pattern. In some embodiments, the thicknesses of the thin films are randomly chosen from a probability distribution function to broaden the spectral profile. In some embodiments, the dielectric stack or a single thin film is coated onto a substrate.

The thin film structure of a dielectric stack (or a thin-film stack, multilayer stack) is designed to have a certain transmittance T and/or reflectance R. Usually, both T and R depend on incident polarization, angle, wavelength, and possibly in the case of stacks with nonlinear elements, intensity. For example, in FIG. 5C, the light that is reflected by the dielectric stack 504 is reflected with a reflectance R that depends on the incident angle θ1 and incident polarization p1 of the light: R=R(θ1, p1). After reflection by the mirror, the light may have its polarization changed into a second polarization p2, and the light that is transmitted by the dielectric stack 504 has a transmittance T that depends on the second polarization p2 and the second incident angle θ2: T=T(θ2, p2). In both cases the wavelength dependance is suppressed. In some embodiments, the mirror 3 comprises a dielectric stack.

The transmittance or reflectance may be calculated as an optimization problem or an inverse problem, whereby the desired function is a target function and the materials, number of layers, and layer thicknesses are optimized to most closely match the target function. In some embodiments, some initial parameters may be fixed as constraints. In some embodiments, the films are periodically layered. In some embodiments, they have random thicknesses chosen according to a probability distribution function to impact how broadband the response is. In some embodiments, a genetic algorithm or AI-based algorithm is used to optimize the response.

Some components of the display system may be polarization dependent. For example, as shown in FIG. 5D, the display system has mounted to its housing 502 an FEC having plurality of specular reflectors including a polarization-dependent beam splitter (PBS) 7 and a QM 31. Light enters the display system through an absorptive polarizer 8 to ensure the light polarized to be reflected by the PBS 7. In some embodiments, the light is polarized at the preparation optics stage. In some embodiments, the light source itself emits polarized light. In some embodiments, the entrance aperture further includes other light profiling optics, such as a directional film or a wave plate. After reflection by the PBS 7, the light is reflected by the QM 31 which also rotates its polarization ninety degrees so as to be then transmitted by the PBS and through an exit aperture, here a Postcavity optics 48, to shield unwanted stray light from exiting the cavity and to prevent ambient light from being redirected to a viewer.

In FIGS. 5A through 5D, light entered the display system from a side and exited at least approximately perpendicular to the entrance direction. In some embodiments, the entrance and exit directions may be parallel. For example, in FIG. 5E, light enters the housing 502 of the display system 305 from the top through an entrance aperture optic 501, which may include a polarizer. The light then passes through a QBQ 30, which transmits the light and rotates its polarization ninety degrees such that the light is reflected by a reflective polarizer 17. The reflected light is subsequently reflected by the QBQ 30 with its polarization again rotated to be transmitted by the reflective polarizer 17 and transmitted by the exit aperture optics 503. In this embodiment, the light is incident twice on the PBS 7 from the same side.

Similarly, in FIG. 5F, light enters the housing 502 of the display system 305 from the top through some entrance optic 501, which may include a polarizer, waveplate, antireflection element, or directional film. In some embodiments, it is curved. In some embodiments, it is tilted at an angle relative to the housing surface. The light is profiled to be polarized to be reflected by the PBS 7, which first directs light to a first QM 31A. The subsequently reflected light is rotated in polarization to be transmitted by the PBS 7, then reflected and rotated again by a second QM 31B. The light is subsequently reflected by the PBS 7 and exits through a Postcavity optics 48, which acts as the exit optic. In some embodiments, the exit optic is curved, or it is flat but tilted at an oblique angle relative to the housing surface as shown. Not that in this embodiment, the light is incident on a specular reflector (here, the PBS 7) more than three times. In other embodiments, the light may be incident on a given specular reflector a plurality of times, such as 2, 3, 4, 5, or 6 times.

In FIG. 5G, a viewer 500 is positioned on a platform 301 to use the display system. Light from a light source 1 is reflected by a relay 304 through an entrance optic 501 and light preparation optics 303. The light then interacts with a plurality of semi-reflectors (here beam splitters 14 and a side mirror 3. Some of the light bounces through the system between a top mirror and an exit optic 503. In some embodiments, the light source 1 emits collimated light or the preparation optics 303 collimates the light. In some embodiments, the light source is a display. In some embodiments, the display is segmented, and different segments travel different paths within the housing 502. The effect is to broaden the image size or create a multifocal display.

In FIG. 5H, a light source 1 emits image-forming light to a relay 302, which may be a mirror. The relay then redirects the light into the display system's housing 502 through an entrance optic. Different rays are reflected by different portions of a prismatic specular reflector 506, to direct the light through an aperture 308 to be viewed by a viewer 500 who is lying on a platform. Similarly, in FIG. 5I, light from a light source 1 passes through some preparation optics 303, which may be a lenselt array to collimate the light and direct it towards a relay 304. The light enters the display system 305 through the entrance optic 501 and it is in-coupled into a waveguide 22 by a grating 24. As the light travels through the waveguide, it is out-coupled by a second wave guide 24 and directed through an aperture 308 to be seen as a virtual image by a viewer 500 on the platform 301. In some embodiments, the light has multiple spectral components, e.g., a red (R), blue (B), and green (G) components, and each component has its own set of waveguide/gratings.

Generally, the optical components that create the virtual image, e.g., the specular reflectors in some of the above embodiments, are called collectively the “light-guiding subsystem.” Light-guiding subsystems include FECs, multi-bounce periscope systems, geometric waveguides, a light pipe, light guide, fiber bundle, and the like.

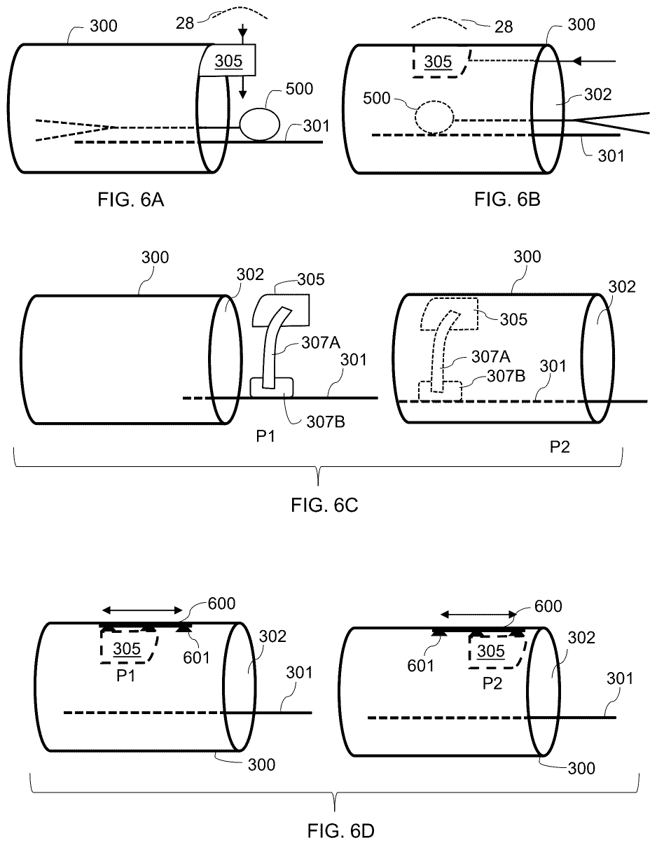

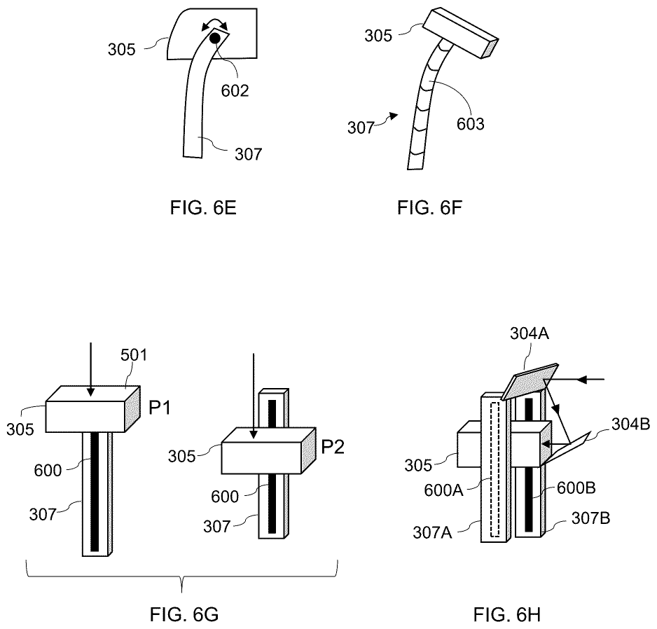

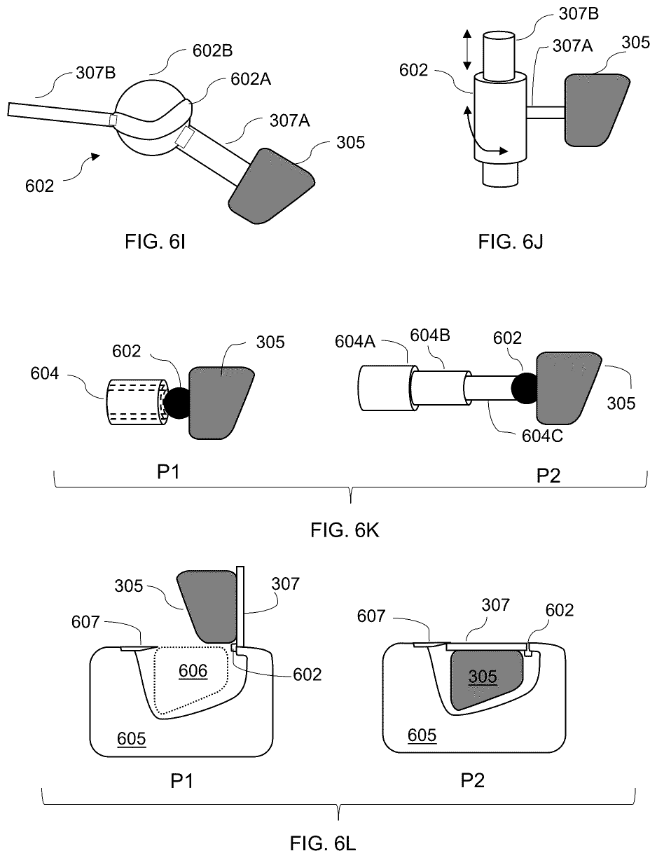

FIGS. 6A through 6H show various ways of integrating the display system to a medical diagnostic tool using various mechanical supports and joints. FIG. 6A shows an embodiment that is characteristic of a feet-first medical diagnostic tool 300, such as an MRI machine. The viewer 500, who is the person to be imaged/diagnostic, lies on a platform 301 which is lower body inside the medical diagnostic tool 300. A display system 305 is mounted to an exterior surface of the tool, and light enters from the top and is directed toward the viewer 500, who sees a virtual image 28.

In FIG. 6B, the medical diagnostic tool 300 may be an MRI machine that is configured for a head-first geometry. The viewer 500 is lying on a platform and enters head first into the tool 300. The display system 305 is mounted to an interior portion of the medical diagnostic tool 300. Image forming light enters through the cavity 300 horizontally and is directed to the viewer 500 who sees a virtual image 28. In some embodiments, the virtual image has a monocular depth such that its focal plane lies outside the medical imaging tool 300. In some embodiments, the monocular depth is such that the focal plane lies at an interior position inside the tool but outside the cavity 302.

In some embodiments, the display system is mounted to a moveable part of the medical diagnostic tool. For example, in FIG. 6C, a medical diagnostic tool 300 includes a platform 301, and the display system 305 is mounted to the platform via a mechanical support, which may include a first mechanical support 307A and a second mechanical support 307B. When the platform is in a first position P1, which may be the case when the patient is being placed onto it, the display system is positioned outside the cavity 302. As the platform and patient move into the cavity 302 into a second position P2, so does the display system 305. This may be the case for a head-first MRI machine, where the patient is lying down with his eyes positioned below the display system 305. In some embodiments, the viewer's head may be next to the second mechanical support 307B. In some embodiments, the first mechanical support 307A is a rigid or flexible arm, and the second mechanical support 307B is a mounting base to attach or remove the display system 305 to the platform.

In FIG. 6D, the display system 305 is mounted to an adjustable track 600, which is positioned within the cavity 302 of a medical diagnostic tool 300, such as within the bore of an MRI machine. The display system 305 may be set in multiple positions, for example, a first position P1 or a second position P2. In some embodiments, a set of notches 601, gradations, fasteners, and the like are included in the adjustable track 600 such that the display system 305 can be moved back and forth into preset positions. In some embodiments of FIG. 6C or 6D, the display system 305 is adjustable to optimize the viewing position.

In FIG. 6E, the display system 305 is mounted to a mechanical support 307 via an adjustable hinge 602 so that the display system's orientation can be changed relative to the mechanical support 307. In some embodiments, the hinge comprises multiple hinges to rotate the display system 305 around multiple axes, such as 1, 2, or 3 axes. In some embodiments, the plurality of axes is mutually perpendicular. Similarly, in FIG. 6F, the display system 305 is mounted to a mechanical support 307, the latter being subdivided into individual support elements 603, such that the entire support is flexible enough to reposition the display system 305 and rigid enough to maintain its position.

All the movable actuators, hinges, flexible arms, ball-and-socket mechanisms, telescoping arms, and the like maybe be called examples of a “mechanical joint.” That is, a mechanical joint is a mechanism that couples two components mechanically whereby the geometry of one component may be changed relative to that of the other.

Adjustment of the display system position or orientation relative to the medical diagnostic tool to which it is mounted can be achieved in a variety of ways. (The “geometry” of an object is its position and orientation, so a change in geometry is a change in either the position or the orientation.) For example, as shown in FIG. 6G, the display system 305 receives light from the top through an entrance optic 501, and the display system itself is mounted to a mechanical support 307 that comprises an adjustable track, so that the display system may be moved into a first position P1 or a second position P2. In this embodiment, note that the position does not affect the directionality of the incident light. In some embodiments, preparation optics may be dynamically modified in accordance with the position to optimize the display content.

Similarly, in FIG. 6H, the display system 305 is mounted to a first and a second mechanical support 307A, 307B, which each have an adjustable track 600A, 600B to move the display system along the track. The two supports may be mounted to either side of the display system. In some embodiments, light enters the display system 305 from the side. To continue receiving the display content as the position is changed, a first and second relay 304A, 304B may be used. In some embodiments, the first relay 304A is mounted to a fixed portion of either or both supports 307A, 307B; and a second relay 304B is mounted to an exterior part of the display system 305 itself. In some embodiments, the relays may be adjusted to optimize the viewing content.

Still other adjusting mechanisms allow the display system's geometry to be changed relative to a mechanical support or to the medical diagnostic tool. For example, FIG. 6I a ball joint 602, which may comprise a ball 602B and a socket 602A, such that a first support 307A connected to the display system 305 may rotate into an arbitrary angle relative to a second support 307B that may be fixed to the medical diagnostic tool. Similarly, as shown in FIG. 6J, the display system 305 is connected to a first support 307A, which is coupled to a rotating hinge 602 that is free to rotate the display round an axis parallel to a second mechanical support 307B. Further vertical position adjustments along the short of the second mechanical support 307B allow for changing the height of the display system.

Shown in FIG. 6K is a telescoping system. The display system 305 is fixed via a hinge 602 to a telescoping actuator 604 that consists of multiple parts. IN a first position P1, the parts are nested and retracted, whereas in a second position P2, the telescoping system is expanded along three parts 604A, 604B, 604C to change the extension of the display system.

In some embodiments, the display system is hidden from view when not being used. In FIG. 6L, when the display system 305 is usable it is in position P1, fixed to a mechanical support 307, which is coupled via a hinge 602 to a subsection 605 of a medical diagnostic tool. The hinge can be rotated to move the display system into a second position P2, where it is closed off into a cavity 606 of the subsection 605. In some embodiments a rubber, fiber, or plastic edge 607 is used to prevent dust or other particles from entering the cavity 606 when the display system is not in use.

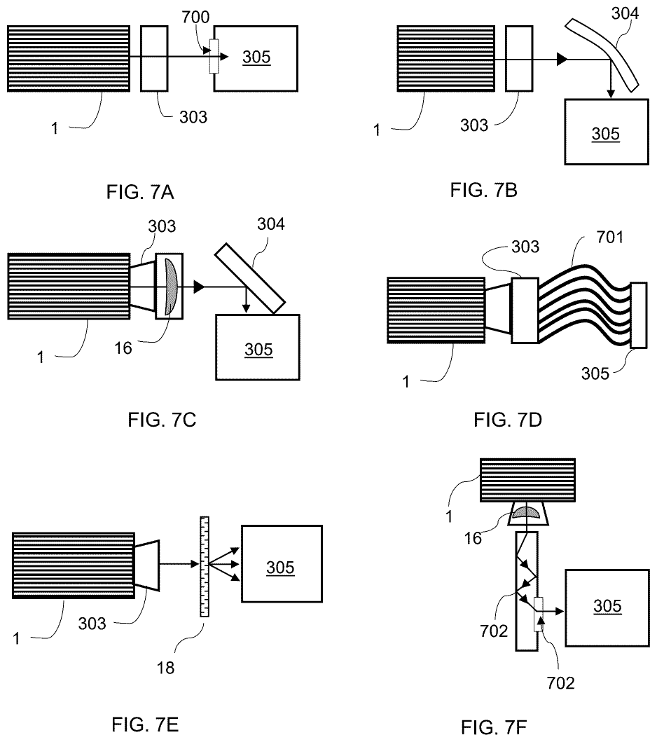

FIGS. 7A through 7F show various mechanisms to generate image-forming light to direct into a display system. Generally, as shown in FIG. 7A, a light source 1 produces or emits light through preparation optics 303, which is then directed into the display system 305 via any of the embodiments described herein, directed through the entrance aperture surface 700. The light source 1 is outside the diagnostic imaging tool. In some embodiments, it is physically separated from the display system. The distance between he light source 1 and the entrance aperture 700 is greater than 0.5 m, 1 m, 1.5 m, 2 m, 3 m, 5 m, 8 m, or 10 m.

In some embodiments, the preparation optics 303 includes a polarizer and a wave plate to impact the polarization of the light before it enters the display system 305. In some embodiments, the preparation optics 303 further includes an LC plate such that the polarization may be modulated dynamically. In some embodiments, the LC plate is a part of the display system. In some embodiments, the LC plate is electromagnetically shielded from the diagnostic tool.

In some embodiments, as shown in FIG. 7B, the light source 1 directs light through the preparation optics 303, after which it is directed into the display system 305 via a relay 304, such as a mirror. In some embodiments, the relay is curved. In some embodiments, the image is precompensated to mitigate any distortion that a curved reflector or relay would produce.

In some embodiments, such as in FIG. 7C, the light source 1 sends light through preparation optics 303, which includes a lens group 16, for example a lens to collimate the image light and direct it to a relay 304 and into the display system 305. The collimation may occur within the light source itself or as a structure added on as shown in the figure. In some embodiments, the lens group 16 only approximately collimates the light. The lens group diameter may be approximately as wide as the exit aperture of the light source 1 and placed approximately one focal length away. In some embodiments, the lens group 16 is used to image the light onto a secondary surface farther along the optical path.

In FIG. 7D, the light source emits light through preparation optics 303, which then directs segments or pixels into an image guide 701. The image guide may be a collection of dielectric guides, waveguides, optical fiber, and the like, and the light exits the image guide 701 and directs the light to the display system 305. In some embodiments, the preparation optics includes an angular profiling layer such as a lenslet array to help collimate or in-couple the light into the elements of the image guide 701. In some embodiments, the light exits the image guide and travels a distance in free space before being incident on the display system.

In FIG. 7E, a light source 1 emits imaging forming light, which may pass through preparation optics 303. The light then lands on a diffuser 18, such as a diffusing screen. In some embodiments, the diffuser is used in transmission mode, such that the transmitted light is directed or scattered onto the entrance 700 of the display system. In other embodiments, the diffuser is used in reflection mode, and the reflected light is incident on the display system. For example, the preparation optics 303 m may include a lens group to image the light onto the diffuser 18, which then acts as the image source location for the display system.

In FIG. 7F, the light source 1 emits light through a lens group 16, which may be a lens to image or collimate light onto a geometric guide 702. The geometric guide may be a thin dielectric slab, thicker than a wavelength of the light, and the light bounces multiple times as it travels along it. The light then exits, either through an exit aperture 702 or through a surface grating and is projected onto the display system 205 to produce a virtual image.

The light source is arbitrarily engineered. It may be a display panel, a display system, a primary virtual display system, a headset or other ARVR device, a laser projector, DLP, LCD, LCoS, SLM DMD, and the like. In some embodiments, the light is coherent light. In some embodiments, it is incoherent light. In some embodiments, it is white light.

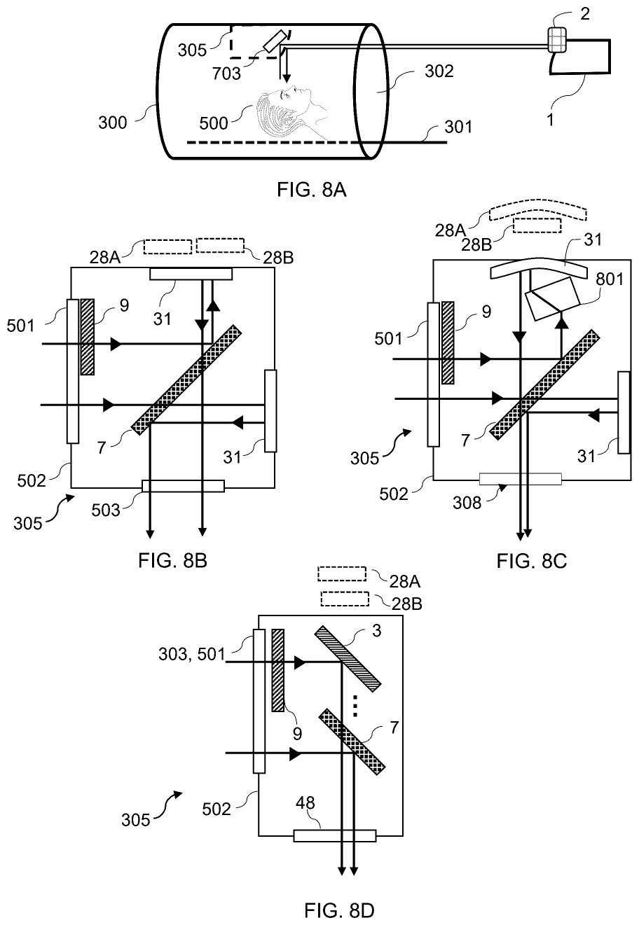

FIGS. 8A through 8D show some auxiliary embodiments of the display system. In FIG. 8A, a light source 1 directs light into a display system 305 through a cavity 302 of a medical diagnostic tool 300, such that a viewer 500 lying on a platform 301 sees a virtual image. A sensor 2 is mounted to the light source 1 (or to another part of the computational system that coordinates the image generation). In some embodiments the sensor is an active sensor in that it emits a signal and detects reflections of the signal. In some embodiments, the sensor is a camera sensor, a camera, or a camera array. Light, or active sensor emission is reflected to and from the viewer 500 by way of a reflector 703. In some embodiments, the sensor is an active infrared sensor. In some embodiments, the sensor is an optical microphone. In some embodiments, the sensor detects information about the eye gaze, head geometry, or other feature of the viewer 500 and feeds that information into the electron control of the light source 1 to impact the image. In some embodiments, the sensor emission and detection channels are not free space but guided along a non-magnetic channel, such as a fiber optic link. In some embodiments, the eye tracking or head tracking is used to assist in maintaining stereoscopic images for the following embodiment.

In FIG. 8B, light enters the display system 305 through an entrance aperture optic 501 which may include a polarizer. Over a portion of this optic is a half-wave plate (HWP) 9, such that a portion of the light image-forming light is a first polarization, and another portion is a perpendicular polarizer. The result is that the first portion is reflected by the PBS 7, reflected and rotated in polarization by a QM 31, transmitted through the PBS 7 and transmitted through an exit aperture optic 503. The other portion of light, being of an orthogonal polarization is first transmitted through the PBS, reflected and rotated in polarization by a QM 31 and reflected by the PBS 7 to exit the housing 502 through the exit aperture optic 503. Each person forms a respective first and second virtual image 28A, 28B. In some embodiments, the first and second virtual images are viewable in a first and second eyebox, such that, for example, a viewer's left eye sees the first virtual image, and a viewer's right eye sees the second virtual image. The two images may form a stereoscopic pair, such that the viewer experiences stereopsis, or binocular depth cues through the vergence of his eyes. In addition, the viewer will perceive the accommodation depth cue caused by the monocular depth of the virtual images. In some embodiments, the two QMs 31 are curved to form concentric-lightfield versions of stereoscopic/monocular virtual images.

In FIG. 8C, image-forming light enters a display system 305 through an entrance aperture optic 501, which includes a polarizer, such as an absorptive polarizer. A portion of the light also travels through a HWP 9 such that a first portion is a first polarization, and the second portion is an orthogonal polarization. The first portion travels through a PBS, is reflected and rotated by a QM 31, reflected by the same PBS 7, and exits the housing 502 through an aperture 308. The other portion is first reflected by the PBS 7, travels through a path-length adjuster 801, is reflected and rotated in polarization by a QM 31, which may be curved, transmitted through the PBS 7 and exits through the aperture 7. The path-length adjuster shifts the light such that both portions of light overlap to create a multifocal image, which consists of at least two virtual images 28A, 28B at different focal planes. The path-length adjuster may be a slab of dielectric material of a certain refractive index, a small geometric light guide to redirect the light rays, a diffraction grating, a nanostructure, or a metasurface. In some embodiments, only the light traveling towards the QM 31 interacts with the path-length adjuster 801, whereas the return light does not.

Last, FIG. 8D, a multifocal image consisting of a first and second virtual image 28A, 28B, each at a different focal length (i.e., each with a different monocular depth) is formed as follows. Image-forming light enters the display system 305 through an entrance aperture optic 501 and/or preparation optics 303, which may include a polarizer. A portion of the polarized light is rotated into an orthogonal polarization by a HWP 9. That portion of light is reflected by a mirror 3, transmitted through a PBS 7, and exits the housing 502n through a Postcavity optics 48. The other portion of light is immediately reflected by the PBS 7 and exits the display system 305. Each portion corresponds to image content in a given focal plane. In principle, more than two focal planes can be produced with a plurality of PBSs or other semi-reflectors and a further segmentation of the image-forming light.

It is also possible to integrate the embodiments of this invention with other optical elements, such as parallax barriers, polarization shutters, or lenticular arrays to send different images to different eyes. In some embodiments, this is aided with an eye tracking module, and in some embodiments, the other optical elements are worn as a headset. These systems then may produce both monocular depth cues and stereoscopic depth cues to trigger accommodation and vergence binocular vision.

Although the invention has been explained in relation to its preferred embodiments, it is to be understood that many other modifications and variations can be made without departing from the spirit and scope of the invention as hereinafter claimed.

In this document, the terms “machine readable medium,” “computer readable medium,” and similar terms are used to refer to non-transitory mediums, volatile or non-volatile, that store data and/or instructions that cause a machine to operate in a specific fashion. Common forms of machine-readable media include, for example, a hard disk, solid state drive, magnetic tape, or any other magnetic data storage medium, an optical disc or any other optical data storage medium, any physical medium with patterns of holes, a RAM, a PROM, EPROM, a FLASH-EPROM, NVRAM, any other memory chip or cartridge, and networked versions of the same.

These and other various forms of computer readable media may be involved in carrying one or more sequences of one or more instructions to a processing device for execution. Such instructions embodied on the medium, are referred to as “instructions” or “code.” Instructions may be grouped in the form of computer programs or other groupings. When executed, such instructions may enable a processing device to perform features or functions of the present application as discussed herein.

In this document, a “processing device” may be implemented as a single processor that performs processing operations or a combination of specialized and/or general-purpose processors that perform processing operations. A processing device may include a CPU, GPU, APU, DSP, FPGA, ASIC, SOC, and/or other processing circuitry.

The various embodiments set forth herein are described in terms of exemplary block diagrams, flow charts and other illustrations. As will become apparent to one of ordinary skills in the art after reading this document, the illustrated embodiments and their various alternatives can be implemented without confinement to the illustrated examples. For example, block diagrams and their accompanying description should not be constructed as mandating a particular architecture or configuration.

Each of the processes, methods, and algorithms described in the preceding sections may be embodied in, and fully or partially automated by, code components executed by one or more computer systems or computer processors comprising computer hardware. The processes and algorithms may be implemented partially or wholly in application-specific circuitry. The various features and processes described above may be used independently of one another or may be combined in several ways. Different combinations and sub-combinations are intended to fall within the scope of this disclosure, and certain method or process blocks may be omitted in some implementations. Additionally, unless the context dictates otherwise, the methods and processes described herein are also not limited to any particular sequence, and the blocks or states relating thereto can be performed in other sequences that are appropriate, or may be performed in parallel, or in some other manner. Blocks or states may be added to or removed from the disclosed example embodiments. The performance of certain operations or processes may be distributed among computer systems or computers processors, not only residing within a single machine, but deployed across a number of computational resources.

As used herein, the term “or” may be constructed in either an inclusive or exclusive sense. Moreover, the description of resources, operations, or structures in the singular shall not be read to exclude the plural. Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps.

Terms and phrases used in this document, and variations thereof, unless otherwise expressly stated, should be constructed as open ended as opposed to limiting. Adjectives such as “conventional,” “traditional,” “normal,” “standard,” “known,” and terms of similar meaning should not be constructed as limiting the item described to a given time period or to an item available as of a given time, but instead should be read to encompass conventional, traditional, normal, or standard technologies that may be available or known now or at any time in the future. The presence of broadening words and phrases such as “one or more,” “at least,” “but not limited to” or other like phrases in some instances shall not be read to mean that the narrower case is intended or required in instances where such broadening phrases may be absent.

What is claimed:

1. A system comprising:
a light guiding subsystem including a plurality of nonmagnetic specular reflectors;
a housing to secure the light guiding subsystem;
an entrance aperture in the housing to direct light from an external light source into the light guiding subsystem; and
an exit aperture in the housing to direct light into a headbox, wherein the specular reflectors are non-magnetic, and the light forms a virtual image visible within the headbox.

2. The system of claim 1, wherein the light source is greater than 0.5 meters from the entrance aperture.

3. The system of claim1, further comprising a mechanical joint to mount the housing to a medical imaging device.

4. The system of claim 3, wherein the medical imaging device is a magnetic resonance imaging (MRI) system, the housing positioned inside a bore of the MRI system.

5. The system of claim 1, wherein the headbox spans a lateral dimension of at least 10 cm.

6. The system of claim 1, wherein at least one specular reflector among the plurality of nonmagnetic specular reflectors comprises a multilayer dielectric film.

7. The system of claim 1, wherein at least one specular reflector among the plurality of nonmagnetic specular reflectors is a curved specular reflector.

8. The system of claim 1, wherein a subset of the plurality of nonmagnetic specular reflectors is polarization dependent.

9. The system of claim 1, further comprising an aperture optic disposed at the exit aperture.

10. The system of claim 1, further comprising an entrance aperture optic disposed at the entrance aperture.

11. The system of claim 1, wherein a monocular depth of the virtual image is at least 1.5 times a distance between the headbox and the exit aperture.

12. The system of claim 1, further comprising a collimating optic to collect the light from the light source onto the entrance aperture.

13. The system of claim 4, wherein in the mechanical joint is adjustable, such that a position of the headbox is variable.

14. The system of claim 1, wherein the image is a multifocal image.

15. The system of claim 2, further comprising a reflector external to the housing to receive the light and redirect it to the entrance aperture.

16. A system comprising:
a light guiding subsystem including a diffractive waveguide and a grating;
a housing to secure the light guiding subsystem;
an entrance aperture in the housing to direct light from an external light source into the light guiding subsystem; and
an exit aperture in the housing to direct light into a headbox, wherein the specular reflectors are non-magnetic, and the light forms a virtual image visible within the headbox.

17. The system of claim 16, further comprising a reflector external to the housing to receive the light and redirect it to the entrance aperture.

18. The system of claim 16, wherein the grating is a surface grating disposed at the exit aperture.

19. The system of claim 16, wherein the waveguide is a plurality of waveguides, each waveguide among the plurality of waveguides accepting light of a respective spectral range.

20. A system comprising:
a field evolving (FEC) cavity having a non-magnetic reflector and a non-magnetic semi-reflector;
a housing to secure the FEC;
an entrance aperture in the housing to direct light from an external light source into the light guiding subsystem; and
an exit aperture in the housing to direct light into a headbox, wherein the light forms a virtual image visible within the headbox.

21. The system of claim 20, wherein the housing is secured to a medical diagnostic tool.
